(19) United States
(12) Patent Application Publication
(10) Pub. No.: US 2024/0190836 A1
(43) Pub. Date: Jun. 13, 2024

(54) XANTHENE DERIVATIVE COMPOUND HAVING HIGH REFRACTIVE INDEX AND (CO)POLYMER COMPRISING SAME

(71) Applicant: RESEARCH & BUSINESS FOUNDATION SUNGKYUNKWAN UNIVERSITY, Suwon-si (KR)

(72) Inventors: Younchul KIM, Seongnam-si (KR); Hend A. Hegazy, Suwon-si (KR); Ju Young CHOI, Suwon-si (KR); Changsik SONG, Seoul (KR)

(73) Assignee: RESEARCH & BUSINESS FOUNDATION SUNGKYUNKWAN UNIVERSITY, Suwon-si (KR)

(21) Appl. No.: 18/372,913

(22) Filed: Sep. 26, 2023

(30) Foreign Application Priority Data
Sep. 26, 2022 (KR) 10-2022-0121847
May 17, 2023 (KR) 10-2023-0064046

(51) Int. Cl. C07D 335/04; C07D 311/96; C08G 18/32; C08G 18/38; C08G 64/16; G02B 1/04

(52) U.S. Cl. CPC C07D 335/04; C07D 311/96; C08G 18/3218; C08G 18/3874; C08G 64/1691; G02B 1/041

(57) ABSTRACT

The present disclosure relates to a xanthene derivative compound with a high refractive index and a manufacturing method therefor. More specifically, provided herein is a compound that has a xanthene-based complex cardo structure and a high refractive index and can be used as a monomer in optical resins requiring a refractive index of 1.7 or higher, and a (co)polymer and an optical lens manufactured therefrom. Given, the xanthene-based complex cardo structure of the compound maximally suppresses the fluidity of the molecular chains and can find applications in the production of resins with high glass transition temperature and excellent thermal stability.

### TECHNICAL FIELD

This invention was carried out with the support of the Ministry of Trade, Industry, and Energy of the Republic of Korea, under the project identification number 1415179963 and project number 20013223. The research project, named “Material Component Technology Development”, with the research task title “Development of Thermoplastic Optical Resin with a Refractive Index Above 1.65 for Smart Device Optical Lenses and Optical Density Above 6.5 Light Absorbing Agent”, was performed by Kookbo Chemical Co., Ltd. under the management of the Korea Institute of Industrial Technology Evaluation and Management from Jan. 1, 2022 to Dec. 31, 2022.

This invention was carried out with the support of the Ministry of Trade, Industry, and Energy of the Republic of Korea, under the project identification number 1415178757 and project number 20013794. The research project, named “Pilot Project for the Establishment of an Industrial Technology Hub Center”, with the research task title “Simultaneous Design Industrial Technology Hub Center for Composite Materials”, was performed by the Sungkyunkwan University Industry-Academic Cooperation Foundation under the management of the Korea Institute of Industrial Technology Evaluation and Management from Jan. 1, 2022 to Dec. 31, 2022.

This application claims priority to and the benefit of Korean Patent Application Number 10-2022-012184, filed in the Korean Intellectual Property Office on Oct. 26, 2022, the entire content of which is incorporated herein by reference.

The present disclosure relates to a xanthene derivative compound with a high refractive index and a copolymer bearing same. More specifically, the present disclosure is concerned with a xanthene derivative having a xanthene-based complex cardo structure, which is a high refractive index monomer available for an optical resin requiring a refractive index of 1.7 or higher, and a (co)polymer bearing same.

### BACKGROUND ART

Conventionally, monomers with improved refractive indices, represented by chemical formulas A (FBPE) to C, have been used to manufacture optical resins with high refractive indices. They have been used in the production of optical resins such as polyurethane, polycarbonate, polyester, acrylic, epoxy, etc. However, they have limitations in applications where high refractive index optical resins are required.

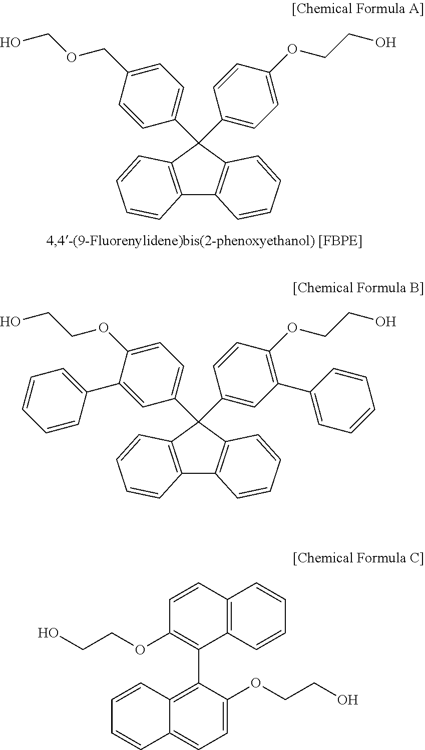 [SMILES: OCCOc1ccc(C2(c3ccc(COCO)cc3)c3ccccc3-c3ccccc32)cc1 ; OCCOc1ccc(C2(c3ccc(OCCO)c(-c4ccccc4)c3)c3ccccc3-c3ccccc32)cc1-c1ccccc1 ; OCCOc1ccc2ccccc2c1-c1c(OCCO)ccc2ccccc12]

Accordingly, there arose a need to develop a novel compound that bears a functional group applicable to resins such as polyester, polycarbonate, polyurethane, polyolefin, epoxy, and acrylic and has a higher refractive index than the conventional commercially available optical resin monomers.

### PRIOR ART DOCUMENT

### Patent Literature

Japanese Patent Number 6648376 (issued on Jan. 20, 2020)

### DISCLOSURE OF INVENTION

### Technical Problem

Thorough and intensive research conducted by the present inventors with the aim of developing a novel compound with a sufficiently high refractive index applicable to optical resins resulted in successfully developing a novel xanthene-based monomer with a refractive index of at least 1.7 which is higher than those of the conventional FBPE by designing molecules in which the electron density is increased by closing the ring between the two aromatic groups at position 9 of the fluorene moiety to increase the electron density and an hetero atom is introduced to improve the refractive index. Moreover, optical resins obtained by applying the novel monomer developed according to the present disclosure to the polymerization of resins such as polyurethane and polycarbonate were evaluated to have an increased refractive index compared to polymers using FBPE. With excellent transparent and thermal properties too, the optical resins are expected to have high application value as functional polymers that requires a high refractive index, high transparency, and high heat resistance.

Therefore, an aspect of the present disclosure is to provide a xanthene derivative compound with a high refractive index and a manufacturing method therefore.

Another aspect of the present disclosure is to provide a polyurethane (co)polymer or a polycarbonate (co)polymer manufactured from the xanthene derivative compound.

A further aspect of the present disclosure is to provide an optical lens including the (co)polymer.

### Solution to Problem

Leading to the present disclosure, thorough and intensive research conducted by the present inventors resulted in preparing novel xanthene derivative compounds with high refractive indices and found that copolymers made of the xanthene derivative compounds have excellent optical properties and thermal resistance.

According to an aspect thereof, the present disclosure provides a xanthene derivative compound having the chemical structure of the following Chemical Formula 1:

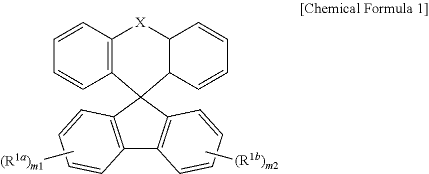

wherein X, R^{1a}, and R^{1b} represent substituents;
X is O, S, or SO₂;
m1 and m2 are each independently an integer of 0 to 4 (with the proviso that m1+m2 is an integer of 1 to 8).

In an embodiment of the present disclosure, R^{1a} and R^{1b} may each be selected from the substituents having the chemical structures of Chemical Formulas 1-1 to 1-3, below:

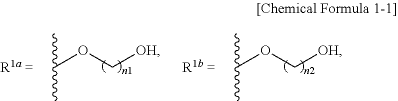

wherein, n1 and n2 are each independently an integer of 1 to 5, with a proviso that n1+n2 is 2 to 10;

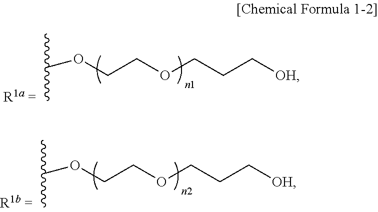

wherein, n1 and n2 are each independently an integer of 1 to 5, with a proviso that n1+n2 is 2 to 10; and

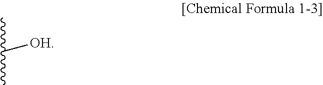 [SMILES: *O]

The xanthene derivative compound according to embodiments of the present disclosure is a monomer compound for optical resins with a refractive index of 1.7. or higher and may have any one of the chemical structures of the following Chemical Formulas 2-1 to 2-3:

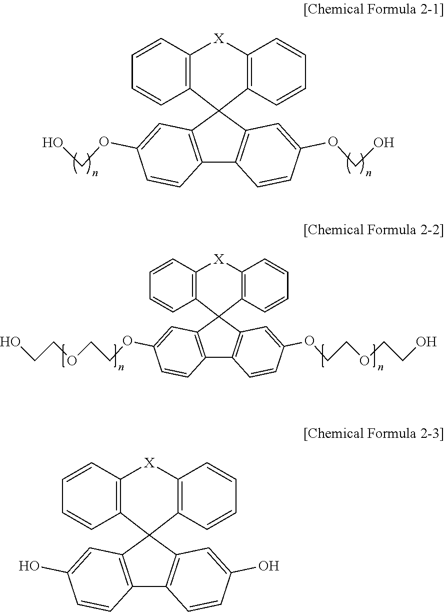

wherein, X is O, S, or SO₂, and n is an integer of 1 to 5.

According to an embodiment, X in Chemical Formula 1 may be SO2. In this regard, the xanthene derivative compound may have the chemical structure of the following Chemical Formula 3-1 or 3-2:

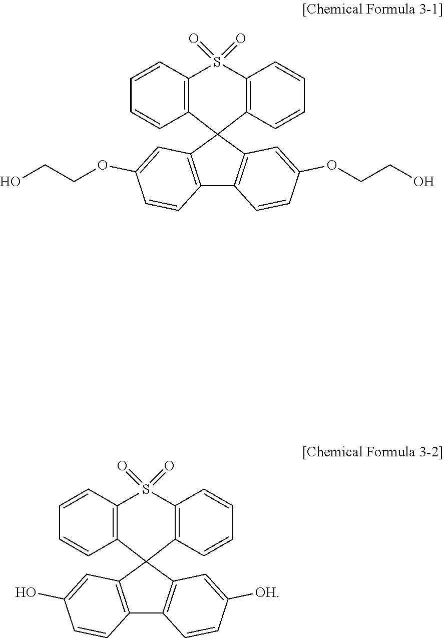 [SMILES: O=S1(=O)c2ccccc2C2(c3cc(OCCO)ccc3-c3ccc(OCCO)cc32)c2ccccc21 ; O=S1(=O)c2ccccc2C2(c3cc(O)ccc3-c3ccc(O)cc32)c2ccccc21]

According to an embodiment, X in Chemical Formula 1 may be O. In this regard, the xanthene derivative compound may have the chemical structure of the following Chemical Formula 4-1 or 4-2:

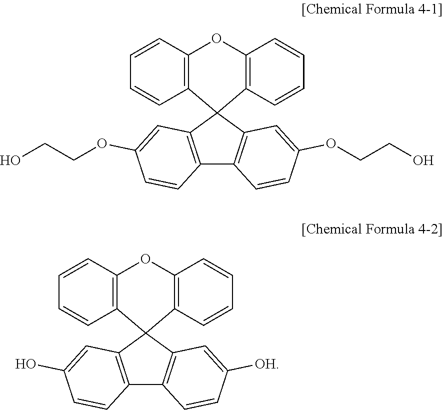 [SMILES: OCCOc1ccc2c(c1)C1(c3ccccc3Oc3ccccc31)c1cc(OCCO)ccc1-2 ; Oc1ccc2c(c1)C1(c3ccccc3Oc3ccccc31)c1cc(O)ccc1-2]

According to an embodiment, X in Chemical Formula 1 may be S. In this regard, the xanthene derivative compound may have the chemical structure of the following Chemical Formula 5-1 or 5-2:

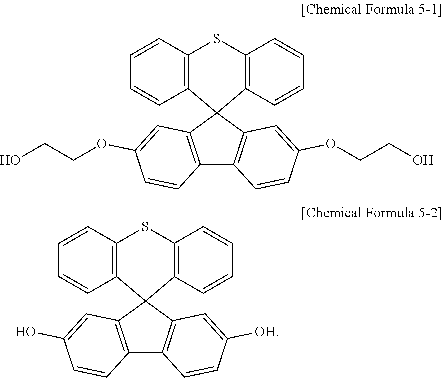

In Chemical Formulas 3-1, 4-1, and 5-1, the hydroxyethyl group, which is the functional group branched at both sides, can be extended in the form of the repeat unit structure of ethyleneoxy by an ethoxy addition reaction.

FIG. 1 is a flow chart illustrating a synthesis method for a xanthene derivative compound according to a first embodiment of the present disclosure.

With reference 1, to FIG. the method for synthesizing a xanthene derivative compound according to an embodiment of the present disclosure includes: a first step of synthesizing 2,7-dimethoxyspiro[fluorene-9,9′-thioxanthene] with the aid of (2-bromophenyl)thiobenzene; a second step of synthesizing spiro[fluorene-9,9′-thioxanthene]-2,7-diol from 2,7-dimethoxyspiro[fluorene-9,9′-thioxanthene]; a third step of synthesizing 2,2′-(spiro[fluorene-9,9′-thioxanthene]-2,7-diylbis(oxy))diethanol from spiro[fluorene-9,9′-thioxanthene]-2,7-diol; and a fourth step of: synthesizing 2,7-bis(2-hydroxyethoxy)spiro[fluorene-9,9′-thioxanthene] 10′,10′-dioxide from 2,2′-(spiro[fluorene-9,9′-thioxanthene]-2,7-diylbis(oxy))diethanol.

In an embodiment, the first step is carried out to synthesize 2,7-dimethoxyspiro [fluorene-9,9′-thioxanthene] by using (2-bromophenyl)thiobenzene as illustrated by the following reaction scheme 1-1:

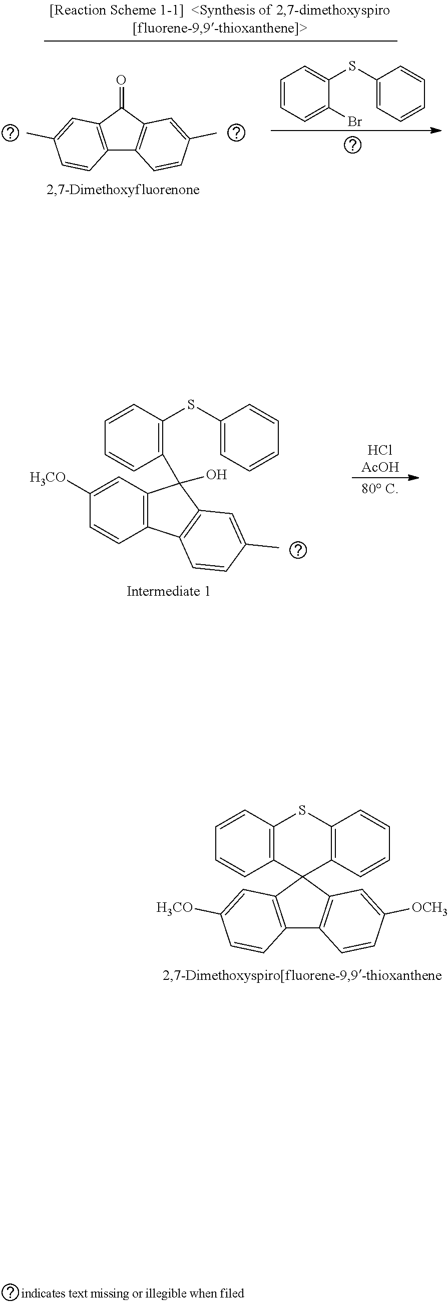

In an embodiment, (2-bromophenyl)thiobenzene is dissolved in an organic solvent, added with drops of nBuLi, and then reacted with 2,7-dimethoxyfluorenone to form the compound of intermediate 1. Subsequently, the compound of intermediate 1 is stirred together with a mixture of hydrochloric acid and acetic acid to synthesize 2,7-dimethoxyspiro[fluorene-9,9′-thioxanthene].

In an embodiment, 2,7-dimethoxyfluorenone may be added in an amount of about 0.6 to 1.0 mole based on 1 mole of (2-bromophenyl)thiobenzene 1 mole, and the reaction may be carried out by stirring while the temperature is increased to room temperature from about −70 to −90° C.

In an embodiment, in order to synthesize 2,7-dimethoxyspiro[fluorene-9,9′-thioxanthene], the compound of intermediate 1 and a mixture solution of hydrochloric acid and acetic acid may be stirred together at a temperature of about 70 to 90° C. for about 8 to 12 hours.

In the second step (S120), spiro[fluorene-9,9′-thioxanthene]-2,7-diol may be synthesized from 2,7-dimethoxyspiro[fluorene-9,9′-thioxanthene] as illustrated by the following Reaction Scheme 1-2.

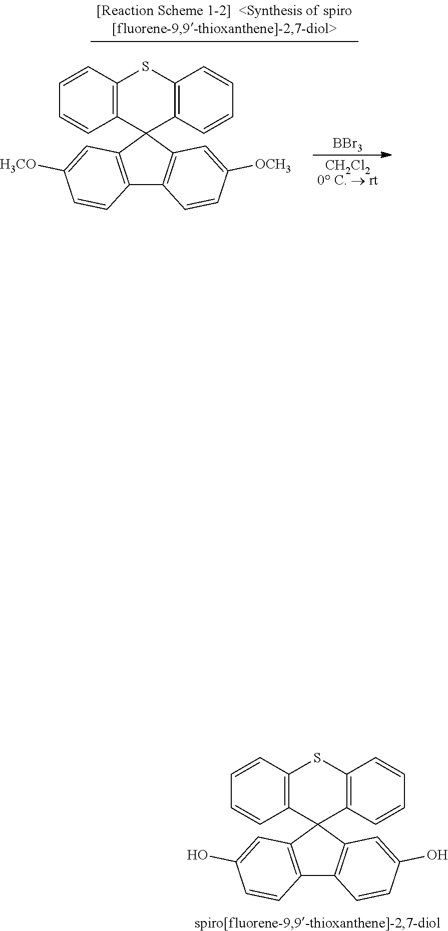

In an embodiment, BBr3 is added dropwise to a solution of 2,7-dimethoxyspiro[fluorene-9,9′-thioxanthene] in dichloromethane (CH₂Cl₂) and stirred to synthesize spiro[fluorene-9,9′-thioxanthene]-2,7-diol.

In the third step (S130), 2,2′-(spiro[fluorene-9,9′-thioxanthene]-2,7-diylbis(oxy))diethanol may be synthesized from spiro[fluorene-9,9′-thioxanthene]-2,7-diol as illustrated by the following Reaction Scheme 1-3.

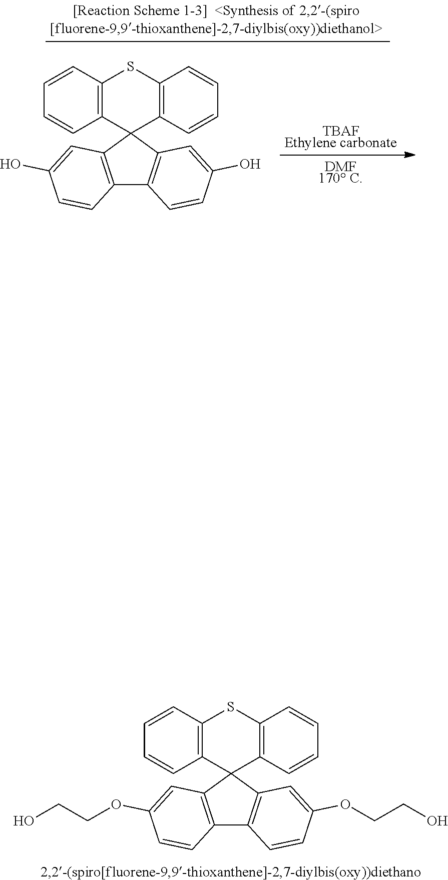 [SMILES: Oc1ccc2c(c1)C1(c3ccccc3Sc3ccccc31)c1cc(O)ccc1-2 ; "DMF170° C." ; "TBAFEthylene carbonate" ; OCCOc1ccc2c(c1)C1(c3ccccc3Sc3ccccc31)c1cc(OCCO)ccc1-2]

In an embodiment, a solution of spiro[fluorene-9,9′-thioxanthene]-2,7-diol in DMF may be added and reacted with ethylene carbonate and TBAF to synthesize 2,2′-(spiro[fluorene-9,9′-thioxanthene]-2,7-diylbis(oxy))diethanol.

In an embodiment, the reaction may be carried out at about 140 to 160° C. for about 2 to 4 hours.

In the fourth step (S140), 2,7-bis(2-hydroxyethoxy)spiro[fluorene-9,9′-thioxanthene] 10′,10′-dioxide is synthesized from 2,2′-(spiro[fluorene-9,9′-thioxanthene]-2,7-diylbis(oxy))diethanol as illustrated by the following Reaction Scheme 1-4.

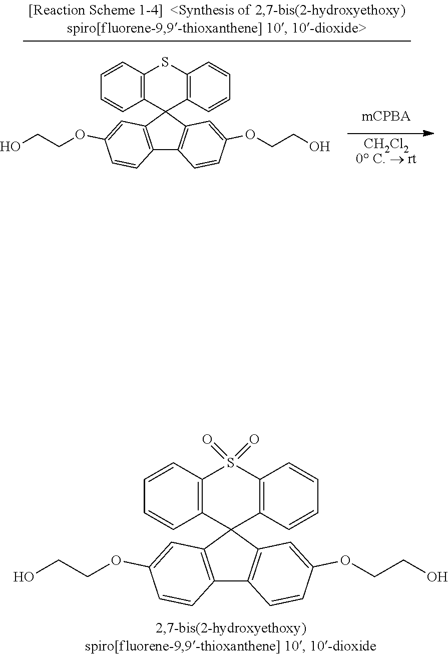

In an embodiment, 2,2′-(spiro[fluorene-9,9′-thioxanthene]-2,7-diylbis(oxy))diethanol is dissolved in an organic solvent and then added and reacted with mCPBA to synthesize 2,7-bis(2-hydroxyethoxy)spiro[fluorene-9,9′-thioxanthene] 10′,10′-dioxide.

In an embodiment, mCPBA may be added in an amount of about 1.5 to 2.5 moles based on 1 mole of 2,2′-(spiro[fluorene-9,9′-thioxanthene]-2,7-diylbis(oxy))diethanol, and the reaction may be carried out for about 4 to 6 hours while stirring.

According to the synthesis method of the first embodiment of the present disclosure, the xanthene derivative compounds of Chemical Formulas 2-1 and 2-3 can be synthesized at high yield with high purity.

FIG. 2 is a flow chart illustrating a synthesis method for a xanthene derivative compound according to a second embodiment of the present disclosure.

Referring to FIG. 2, the method for synthesizing a xanthene derivative compound according to the second embodiment of the present disclosure includes: a first step (S210) of synthesizing 2,7-dibromospiro[fluorene-9,9′-xanthene] from 2,7-dibromofluorenone; a second step (S220) of synthesizing 2,7-dimethoxyspiro[fluorene-9,9′-xanthene] from 2,7-dibromospiro[fluorene-9,9′-xanthene]; a third step (S230) of synthesizing 2,7-dihydroxyspiro[fluorene-9,9′-xanthene] from 2,7-dimethoxyspiro[fluorene-9,9′-xanthene]; and a fourth step (S240) of 2,2′-(spiro[fluorene-9,9′-xanthene]-2,7-diylbis(oxy))diethanol from 2,7-dihydroxyspiro[fluorene-9,9′-xanthene].

In the first step (S210), 2,7-dibromospiro[fluorene-9,9′-xanthene] is synthesized from 2,7-dibromofluorenone as illustrated by the following Reaction Scheme 2-1:

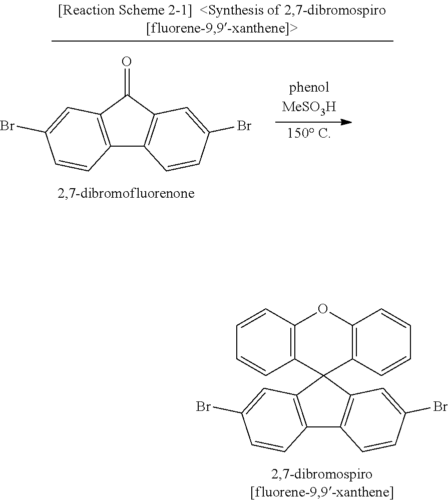 [SMILES: O=C1c2cc(Br)ccc2-c2ccc(Br)cc21 ; Brc1ccc2c(c1)C1(c3ccccc3Oc3ccccc31)c1cc(Br)ccc1-2]

In an embodiment, 2,7-dibromofluorenone and phenol are mixed and the mixture is added and reacted with methanesulfonic acid to synthesize 2,7-dibromospiro[fluorene-9,9′-xanthene].

In an embodiment, phenol may be added in an amount of about 8 to 12 moles and methanesulfonic acid may be added in an amount of about 3 to 5 moles, based on 1 mole of 2,7-dibromofluorenone.

In an embodiment, the reaction may be carried out at about 140 to 160° C. for about 10 to 14 hours in a stirring condition.

In the second step (S220), 2,7-dimethoxyspiro[fluorene-9,9′-xanthene] may be from 2,7-dibromospiro[fluorene-9,9′-synthesized xanthene] as illustrated by the following Reaction Scheme 2-2.

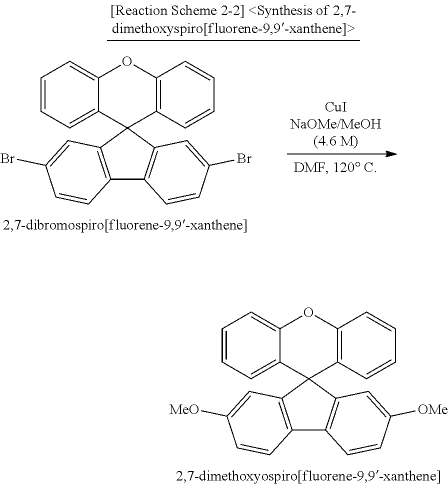 [SMILES: Brc1ccc2c(c1)C1(c3ccccc3Oc3ccccc31)c1cc(Br)ccc1-2 ; COc1ccc2c(c1)C1(c3ccccc3Oc3ccccc31)c1cc(OC)ccc1-2]

In an embodiment, 2,7-dibromospiro[fluorene-9,9′-xanthene], CuI, and DMF are stirred together and mixed in a nitrogen atmosphere, added with NaOMe or MeOH, and then stirred under reflux to synthesize 2,7-dimethoxyspiro[fluorene-9,9′-xanthene].

In an embodiment, CuI may be used in an amount of about 3 to 5 moles, based on 1 mole of 2,7-dibromospiro[fluorene-9,9′-xanthene].

In an embodiment, the stirring under reflux may be conducted at about 110 to 130° C. for about 22 to 26 hours.

In the third step (S230), 2,7-dihydroxyspiro[fluorene-9,9′-xanthene] may be synthesized from 2,7-dimethoxyspiro[fluorene-9,9′-xanthene], as illustrated by the following Reaction Scheme 2-3.

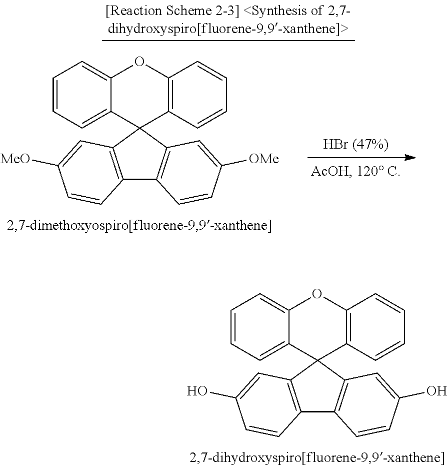 [SMILES: COc1ccc2c(c1)C1(c3ccccc3Oc3ccccc31)c1cc(OC)ccc1-2 ; Oc1ccc2c(c1)C1(c3ccccc3Oc3ccccc31)c1cc(O)ccc1-2]

In an embodiment, 2,7-dihydroxyspiro[fluorene-9,9′-xanthene], Glacial acetic acid and HBr may be mixed and stirred together under reflux to synthesize 2,7-dihydroxyspiro[fluorene-9,9′-xanthene].

In an embodiment, HBr may be used in an amount of about 8 to 10 moles, based on 1 mole of 2,7-dihydroxyspiro[fluorene-9,9′-xanthene].

In an embodiment, the stirring under reflux may be conducted at about 110 to 130° C. for about 46 to 50 hours.

In the fourth step (S240), 2,2′-(spiro[fluorene-9,9′-xanthene]-2,7-diylbis(oxy)) diethanol may be synthesized from 2,7-dihydroxyspiro[fluorene-9,9′-xanthene] as illustrated by the following Reaction Scheme 2-4.

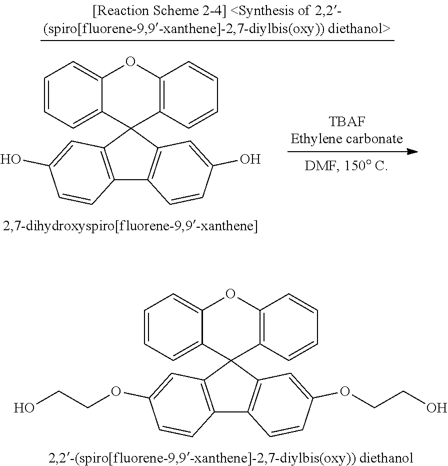 [SMILES: Oc1ccc2c(c1)C1(c3ccccc3Oc3ccccc31)c1cc(O)ccc1-2 ; OCCOc1ccc2c(c1)C1(c3ccccc3Oc3ccccc31)c1cc(OCCO)ccc1-2]

In an embodiment, 2,7-dihydroxyspiro[fluorene-9,9′-xanthene], ethylene carbonate, TBAF, and DMF are mixed and stirred under reflux to synthesize 2,2′-(spiro[fluorene-9,9′-xanthene]-2,7-diylbis(oxy))diethanol.

In an embodiment, ethylene carbonate and TBAF may be used in an amount of about 2 to 2.5 moles and about 0.001 to 0.05 moles, respectively, based on 1 mole of 2,7-dihydroxyspiro[fluorene-9,9′-xanthene].

According to the synthesis method of the second embodiment of the present disclosure, the xanthene derivative compounds of Chemical Formulas 3-1 and 3-2 can be synthesized at high yield with high purity.

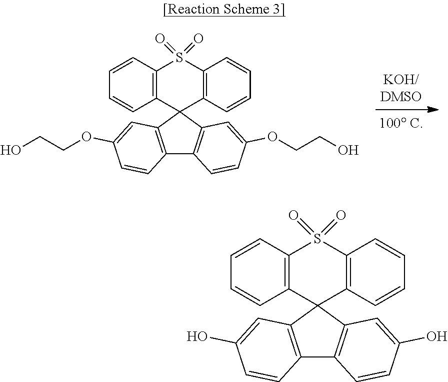 [SMILES: O=S1(=O)c2ccccc2C2(c3cc(OCCO)ccc3-c3ccc(OCCO)cc32)c2ccccc21 ; O=S1(=O)c2ccccc2C2(c3cc(O)ccc3-c3ccc(O)cc32)c2ccccc21]

In an embodiment, 2,7-bis(2- hydroxyethoxy)spiro[fluorene-9,9′-thioxanthene] 10′,10′-dioxide, and KOH may be mixed and stirred under reflux to synthesize 2,7-bis(2-hydroxy)spiro[fluorene-9,9′-thioxanthene] 10′,10′-dioxide.

In an embodiment, KOH may be used in an amount of 3 to 10 moles, based on 1 mole of 2,7-bis(2-hydroxyethoxy)spiro[fluorene-9,9′-thioxanthene] 10′,10′-dioxide.

The compound of the present disclosure is a high-refractive index monomer with a xanthene complex cardo structure, which can be used for an optimal resin requiring a refractive index of 1.7 or higher.

As used herein, the term “cardo” compound refers to a compound structured to have a cyclic side group grafted to the backbone thereof. Cardo compounds have the structural feature of bulky lateral groups present in the polymer backbone, which gives them severe rotational hindrance to the backbone, resulting in very high heat resistance (high glass transition temperature) and excellent processability.

According to another aspect of the present disclosure, the xanthene derivative compound of the present disclosure retains four phenyl groups, which can enhance or improve various properties including optical properties. Therefore, the xanthene derivative compound of the present disclosure can be advantageously used as a resin component, additive, etc. In addition, when applied to a resin, the xanthene derivative compound of the present disclosure, which has a plurality of hydroxy groups, can efficiently improve the properties of the resin.

In one embodiment of the present disclosure, the resin component is either (i) a resin that includes a xanthene-based compound represented by Chemical Formula 1 as a monomer, or (ii) a resin composed of the xanthene-based compound and a resin.

In one embodiment of the present disclosure, no particular limitations are imparted to the resins including the resin component (i.e., the resin component (i) or (ii)), as exemplified by conventional thermoplastic resins, and thermosetting resins (or photocurable resins). The resins may be used alone or in combination.

Examples of thermoplastic resins include olefinic resins (polyethylene, polypropylene, polymethylpentene, amorphous polyolefins, etc.), halogen-containing vinyl resins (chlorinated resins such as polyvinyl chloride, fluorinated resins, etc.), acrylic resins, styrene resins (polystyrene, acrylonitrile-styrene resins, etc.), polycarbonate resins (bisphenol A-type polycarbonate, etc.), polyester resins (polyethylene terephthalate, polybutylene terephthalate, polycyclohexane dimethyl terephthalate, polyethylene naphthalate, etc.), polyalkylene arylate resins, polyarylate resins, liquid crystal polyesters, etc.), polyacetal resins, polyamide resins (polyamide 6, polyamide 66, polyamide 46, polyamide 6 T, polyamide MXD, etc.), polyphenylene ether resins (modified polyphenylene ether, etc.), polysulfone resins (polysulfone, polyethersulfone, etc.), polyphenylene sulfide resins (polyphenylene sulfide, etc.), polyimide resins (polyetherimide, polyamideimide, polyaminobismaleimide, bismaleimide triazine resins, etc.), polyether ketone resins (polyether ketone, polyether ether ketone, etc.), thermoplastic elastomers (polyamide elastomers, polyester elastomers, polyurethane elastomers, polystyrene elastomers, polyolefin elastomers, polydiene elastomers, polyvinyl chloride elastomers, fluorine-based thermoplastic elastomers, etc.). Thermoplastic resins may be used alone or in combination with two or more resins.

Examples of thermosetting resins include phenolic resins, amino resins (urea resins, melamine resins, etc.), furan resins, unsaturated polyester resins, epoxy resins, thermosetting polyurethane resins, silicone resins, thermosetting polyimide resins, and diallyl phthalate resins, vinyl ester resins (resins obtained by the reaction of epoxy resins with (meth) acrylic acid or derivatives thereof, resins obtained by the reaction of polyfunctional phenols with glycidyl (meth) acrylates, etc.). Also included in thermosetting resins (or photocurable resins) are multifunctional (meth) acrylates, vinyl ethers (such as divinyl ether obtained by the reaction of diols with acetylene, etc.). Thermosetting resins may be used alone or in combination.

In addition, the thermosetting resins (or photocurable resins) may contain initiators, reactive diluents, hardeners, and curing accelerators, depending on the type thereof. For example, resin compositions containing epoxy resins or urethane resins may contain amine-based hardeners, while resin compositions containing unsaturated polyester resins or vinyl ester resins may contain initiators (such as peroxides), and polymerizable monomers ((meth)acrylic acid esters, styrene, etc., as reactive diluents).

The resin components (i) or the resin components (ii) of the present disclosure may be used alone or in combination.

In addition, the resin component (i) containing the components (monomers) preferably has a resin framework composed of the xanthene derivative compound and may be prepared into a polymer while the xanthene derivative compound being used fully or partially instead of a polymer component (e.g., polyols, such as diols, etc.). For instance, resins use that polyol components (particularly diol components) as polymerization components or constituents (polyester resins, polyurethane resins, epoxy resins, vinyl ester resin, polyfunctional (meth)acrylate, (poly)urethane (meth)acrylate, (poly)ester (meth)acrylate, vinyl ester, etc.) may employ the xanthene derivative compound in substitution for all or part of the polyol component).

In the resin component (i), the xanthene derivative compounds may be used alone or in combination.

Examples of resins (or resin components) containing the preferable resin component include polyester resins, polyurethane resins (thermoplastic or thermosetting polyurethane resins), polycarbonate resins, acrylic resins (inclusive of thermosetting or photocurable resins such as polyfunctional (meth)acrylates), epoxy resins, and vinyl ethers. In addition, also preferable are resins (thermosetting resins) containing aromatic rings (benzene rings), for example, aromatic polycarbonate resins (bisphenol A polycarbonates, etc.), polyester resins (polyalkylene arylate resin; employing as polymerization components aromatic dicarboxylic acid (terephthalic acid, etc.) and aromatic diol (bisphenol, bisphenol A, xylene glycol, alkylene oxide adducts thereof, etc.)), polysulfone resins (polysulfone, polyether sulfone, etc.), polyphenylene sulfide resins (polyphenylene sulfide, etc.).

Below, resins (or resin components (i)) containing the xanthene derivative compound represented by Chemical Formula 1 as a monomer component (polymerization component, constituent, copolymerization component) will be explained in relation to representative resins (or resin components).

### (1) Polyester Resin

A polyester resin containing the xanthene derivative compound as a polymerization component may be obtained by a reaction between the xanthene derivative compound and a dicarboxylic acid component. Polyester resins include polyarylate resins employing aromatic dicarboxylic acid as polymerization components in addition to saturated or unsaturated polyester resins.

Polyol components (especially diol components) in polyester resins can be composed of a combination of the xanthene derivative compound and other diols. These diol components (or diols) include alkylene glycols (for example, linear or branched C2-12 alkylene glycols, such ethylene glycol, propylene glycol, trimethylene as, glycol, 1,3-butanediol, tetramethylene glycol, hexanol, neopentyl glycol, octane diol, and decane diol), (poly) oxyalkylene glycols (for example, diethylene glycol, triethylene glycol, dipropylene glycol, and C2-4 alkylene glycols), cyclic diols (for example 1,4-cyclohexanediol, 1,4-cyclohexanedimethanol, 2,2-bis(4-hydroxycyclohexyl) propane or alkylene oxide adducts thereof (e.g., 2,2-bis(4-(2-hydroxyethoxy)cyclohexyl)propane), and aromatic diols (for example, biphenol, 2,2-bis(4-hydroxyphenyl) propane (bisphenol A), bisphenol AD, bisphenol For alkylene oxides (C2-3 alkylene oxides) thereof (2,2-bis(4-(2-hydroxyethoxy)phenyl)propane), and xylene glycols). These diols can be used alone or in combination.

Preferable diols are linear or branched C2-10 alkylene glycols, especially C2-6 alkylene glycols (for example, linear or branched C2-4 alkylene glycols such as ethylene glycol, propylene glycol, 1,4-butanediol, etc.). Ethylene glycol is often used among these diols. By using such diols (for example, ethylene glycol), an improvement may be brought into polymerization reactivity with the concomitant impartment of flexibility to the resin.

The xanthene derivative compound and the diols may be used, for example, at a ratio (molar ratio) of 100/0 to 50/50, preferably 100/0 to 75/25 (e.g., 100/0 to 70/30) or 100/0-90/10 (e.g., 100/0 to 80/20).

The diol component may be used, as necessary, in combination of polyols such as glycerin, trimethylolpropane, trimethylolethane, or pentaerythritol.

Dicarboxylic acid component in polyester resins may be aliphatic dicarboxylic acid, alicyclic dicarboxylic acid, aromatic dicarboxylic acid, or ester-formable derivatives thereof (e.g., acid anhydrides; acid halides (e.g., acid chlorides) ; lower alkyl esters (e.g., C1-2 alkyl esters), etc.). These dicarboxylic acids may be used alone or in combination.

Examples of the aliphatic dicarboxylic acids include saturated C3-20 aliphatic dicarboxylic acids such as succinic acid, glutaric acid, adipic acid, pimelic acid, suberic acid, azelaic acid, sebacic acid, dodecanedicarboxylic acid, hexadecanedioic acid (preferably, saturated C3-14 aliphatic dicarboxylic acids) ; unsaturated C4-20 aliphatic dicarboxylic acids, such as maleic acid, fumaric acid, citraconic acid, and mesaconic acid (preferably unsaturated C4-14 aliphatic dicarboxylic acids) ; and ester-formable derivatives thereof. In the unsaturated polyester resin, aliphatic unsaturated dicarboxylic acid (e.g., maleic acid or anhydride thereof) may be present at a proportion of, for example, 10-100 mol %, preferably 30-100 mol %, and more preferably 50-100 mol % (e.g., 75-100 mol %), based on the total mole of the resin.

The alicyclic dicarboxylic acid may be exemplified by saturated alicyclic dicarboxylic acids (e.g., C3-10 such as cycloalkane dicarboxylic acids cyclopentane dicarboxylic acid, 1,4-cyclohexane dicarboxylic acid, 1,3-cyclohexane dicarboxylic acid, 1,2-cyclohexane dicarboxylic acid, and cycloheptane dicarboxylic acid), dicarboxylic acids (C3-10 unsaturated alicyclic cycloalkene dicarboxylic acids, such as 1,2-cyclohexene dicarboxylic acid, 1,3-cyclohexene dicarboxylic acid, etc.), polycyclic alkane dicarboxylic acids (di- or tricyclo C7-10 alkane dicarboxylic acids such as bornane dicarboxylic acid, norbornane dicarboxylic acid, adamantane dicarboxylic acid, etc.), polycyclic alkene dicarboxylic acids (di- or tricyclo C7-10 alkene dicarboxylic acids such as bornene dicarboxylic acid, norbornene dicarboxylic acid, etc.), and ester-formable derivatives thereof.

For the aromatic dicarboxylic acid, examples include aromatic C8-16 dicarboxylic acids such as phthalic acid, isophthalic acid, terephthalic acid, naphthalene dicarboxylic acid (e.g., 2,6-naphthalene dicarboxylic acid), 4,4′-diphenyl dicarboxylic acid, diphenyl ether-4,4′-dicarboxylic acid, 4,4′-diphenylmethane dicarboxylic acid, 4,4′-diphenylketone dicarboxylic acid, etc.; and ester-formable derivatives thereof.

If needed, dicarboxylic acid may be combined with polybasic carboxylic acids such as trimellitic acid, pyromellitic acid, etc.

As the dicarboxylic acid component, at least one selected from aliphatic dicarboxylic acids and alicyclic dicarboxylic acids is used, with preference for aliphatic dicarboxylic acids (saturated aliphatic dicarboxylic acids or ester-formable derivatives thereof, particularly, saturated C3-14 aliphatic dicarboxylic acids such as adipic acid, suberic acid, sebacic acid, etc.) or alicyclic dicarboxylic acids (C5-10 cycloalkane dicarboxylic acids such as cyclohexane dicarboxylic acid, etc.).

Furthermore, for polyarylate resins, dicarboxylic acid components that include at least an aromatic dicarboxylic acid, are used. The aromatic dicarboxylic acid may be used in combination with other dicarboxylic acids (aliphatic dicarboxylic acids and/or alicyclic dicarboxylic acids). The ratio of aromatic dicarboxylic/other dicarboxylic acids may range, for example, from 100/0 to 10/90, preferably from 100/0 to 30/70, and more preferably from 100/0 to 50/50.

In polyester resins, the ratio (molar ratio) of the dicarboxylic acid components/the polyol components (diol components, the xanthene derivative compounds, etc.) may generally range from 1.5/1 to 0.7/1 and preferably from 1.2/1 to 0.8/1 (especially about from 1.1/1 to 0.9/1).

In polyester resins, the ratio (molar ratio) of the dicarboxylic acid components/the polyol components (including diol components, the xanthene derivative compounds) may generally range from 1.5/1 to 0.7, preferably from 1.2/1 to 0.8/1, and more preferably from 1.1/1 to 0.9/1.

The polyester resin may have a weight average molecular weight Mw of, for example, 100 to 50×10⁴, preferably 500 to 30×10⁴ (e.g., 1000 to 20×10⁴), and more preferably 3000 to 30×10⁴, as expressed in polystyrene equivalent, but with no limitations thereto. For unsaturated polyester resins, the molecular weight per double bond may be 300 to 1,000, preferably 350 to 800, and more preferably 400 to 700. The terminal groups of the polyester resin may be either hydroxyl groups or carboxyl groups and may be protected by a protective group, as necessary.

Polyester resins may be manufactured by conventional methods. For example, polyester resins can be prepared by condensation between polyol components (particularly diol components) including of the xanthene derivative compound and the dicarboxylic acid components through a direct polymerization method (direct esterification method) or an ester exchange method.

### (2) Polyurethane Resin

The polyol components (diol components) for the polyurethane resin may be composed of the xanthene derivative compound as a monomer alone or in combination with the diols exemplified for the polyester resins. Additionally, diol components that include the xanthene derivative compound as a constituent unit, for example, polyester diols formed by the reaction of the diol component composed of the xanthene derivative compound of Chemical Formula 1 wherein p1=p2=1, with the dicarboxylic acid component, and polyether diols formed by the reaction of the said diol component with alkylene oxide may also be utilized as diol components for the polyurethane resin. The diol components can be used either alone or in combination. Also, diol components may be used in combination with other polyol components such as triols, as necessary.

Based on the entire polyol components (diol components), the content of the xanthene derivative compound may be, for example, 10-100 mol %, preferably 20-80 mol %, and more preferably around 30-70 mol %.

Available as diisocyanate compounds that make up the polyurethane resin, aromatic diisocyanates [paraphenylenediisocyanate, tolylene diisocyanate (TDI), xylene diisocyanate (XDI), tetramethyl xylene diisocyanate (TMXDI), naphthalene diisocyanate (NDI), bis(isocyanatophenyl) methane (MDI), toluene diisocyanate (TODI), 1,2-bis(isocyanatophenyl) ethane, 1,3-bis(isocyanatophenyl) propane, 1,4-bis(isocyanatophenyl) butane, polymeric MDI, etc.]; cycloaliphatic diisocyanates [cyclohexane 1,4-diisocyanate, isophorone diisocyanate (IPDI), hydrogenated XDI, hydrogenated MDI, etc.]; aliphatic diisocyanates [hexamethylenediisocyanate (HDI), trimethyl hexamethylenediisocyanate (TMDI), lysine diisocyanate (LDI), etc.]. These diisocyanate compounds may be used either alone or in combination. If necessary, these diisocyanate compounds can be combined with polyisocyanate compounds (for example, triiocyanates, e.g., aliphatic triisocyanates such as 1,6,11-undecanetriisocyanatomethyloctane and 1,3,6-hexamethylenetriisocyanate, and cycloaliphatic triisocyanates such as bi(cyclohexanetriisocyanate), etc.) and monoisocyanate compounds (e.g., C1-6 alkyl isocyanates such as methyl isocyanate, C5-6 cycloalkyl isocyanates, and C6-10 aryl isocyanates such as phenyl isocyanate, etc.). Multimers and modified derivatives of the polyisocyanate compounds are also included in the isocyanate compounds.

The polyurethane resin can be obtained by reacting a diisocyanate component in an amount 0.7-2.5 moles, preferably 0.8-2.2 moles, and more preferably around 0.9-2 moles per mole of a polyol component (diol component) using a conventional method. In addition, about 0.7-1.1 moles of diisocyanate component may be used per mole of diol component to obtain a thermoplastic resin. Using an excess mole (for example, about 1.5-2.2 moles) of diisocyanate component, a thermosetting resin with a free isocyanate group at the terminal thereof can be achieved.

### (3) Polycarbonate Resin

A polycarbonate resin containing the xanthene derivative compound as a polymer component may be obtained according to conventional methods, for example, by the reaction of a polyol component (especially a diol component) composed of the xanthene derivative compound with phosgene (phosgene method), or by the reaction of a polyol component (diol component) composed of the xanthene derivative compound with a carbonate ester (ester exchange method).

The polyol component (diol component) can be composed of the xanthene derivative compound alone or in combination with other diols (diols exemplified in the polyesters, particularly aromatic diols or cycloaliphatic diols, etc.). The other diols can be used either alone or in combination. Among the other diols, aromatic diols such as bisphenols, especially bisphenol A, AD, F, etc., are preferred. The ratio of the xanthene derivative compound with a hydroxyl group to diols can be selected in the same range as for the polyesters.

The weight average molecular weight of polycarbonate resin is not particularly limited. For instance, the polycarbonate resin may have a weight average molecular weight of 1×10³ to 100×10⁴ (e.g., 1×10⁴ to 100×10⁴), preferably 5×10³ to 50×10⁴ (e.g., 1×10⁴ to 50×10⁴), and more preferably 1×10⁴ to 25×10⁴ (e.g., 1×10⁴ -10×10⁴), but with no limitations thereto.

### (4) Epoxy Resin

The diol component or polyol component in the epoxy resin can be composed of the xanthene derivative compound alone or in combination with other diols (particularly aromatic diols or cycloaliphatic diols, etc.) different from those for the polyester resin. The other diols can be used either alone or in combination. Among the diols, aromatic diols such as bisphenols, especially bisphenol A, AD, F, etc., are preferred. The ratio of the xanthene derivative compound to diols may be selected in the same range as for the polyester resin. Furthermore, the bisphenol xanthene series and other diols, if necessary, may be combined with polyols (e.g., phenol novolac, etc.).

The epoxy resin can be obtained, for example, by reacting at least the xanthene derivative compound with epichlorohydrin. The epoxy resin may have a weight average molecular weight (Mw) of, for instance, 300-30,000, preferably 400-10,000, and more preferably 500-5,000.

### (5) Vinyl Ester Resin

Vinyl ester resin can be obtained by conventional methods, for example, by reacting the epoxy resin (which contains the xanthene derivative compound as a component) with a polymerizable monomer having a carboxyl group (unsaturated monocarboxylic acid). The polymerizable monomer with a carboxyl group may also be used in combination with the polyester resin and dicarboxylic acid (aliphatic dicarboxylic acid, cycloaliphatic dicarboxylic acid, or aromatic dicarboxylic acid (isophthalic acid, terephthalic acid, etc.) as necessary.

As the polymerizable monomer with a carboxyl group, unsaturated monocarboxylic acids can be used. Typically, (meth) acrylic acid may be used as the unsaturated monocarboxylic acid. Other available unsaturated monocarboxylic acids include cinnamic acid, crotonic acid, sorbic acid, maleic monoalkyl esters (e.g., monomethyl maleate). These monomers can be used alone or in combination.

The amount of unsaturated monocarboxylic acid may be in the range of 0.5-1.2 moles, preferably 0.7-1.1 moles, and more preferably 0.8-1 mole per mole of epoxy groups in the epoxy resin.

The vinyl ester resin can also be obtained by reacting the xanthene derivative compound with glycidyl (meth)acrylate. The glycidyl (meth)acrylate may be used in an amount of, for instance, 1-3 moles and preferably 1-2 moles per mole of the xanthene derivative compound.

### (6) Acrylic Resin

The monomers for the acrylic resin can be obtained by reacting the xanthene derivative compound with a polymerizable monomer bearing a carboxyl group. Typically, unsaturated monocarboxylic acids, especially (meth)acrylic acid can be used as the polymerizable monomer with a carboxyl group. Other available unsaturated monocarboxylic acids include cinnamic acid, crotonic acid, sorbic acid, maleic monoalkyl esters (e.g., monomethyl maleate). These monomers can be used alone or in combination.

Acrylic resin may be a homopolymer or copolymer of a (meth)acrylic monomer that has the xanthene frame work, or it can be a copolymer of a (meth)acrylic monomer having a xanthene frame work and a copolymerizable monomer. Examples of the copolymerizable monomer include: carboxyl-containing monomers such as (meth)acrylic acid, maleic acid, and anhydrous maleic acid; (meth)acrylic esters [e.g., (meth)acrylic acid C1-6 alkyl esters such as (meth)acrylic acid methyl, etc. ]; vinyl cyanide such as (meth)acrylonitrile, etc.; aromatic vinyl monomers such as styrene; vinyl esters of carboxylic acids such as vinyl acetate; and α-olefins such as ethylene, propylene, etc.

These copolymerizable monomers may be used alone or in combination.

Furthermore, the monomer having multiple (meth)acryloyl groups obtained from the reaction of the xanthene derivative compound with the polymerizable monomer having a carboxyl group can be used as an acrylic resin (i.e., thermosetting acrylic resin or oligomer (resin precursor)).

Also, the resin component (ii) can be manufactured or formulated by mixing the xanthene derivative compound with a resin (and additives if needed).

The mixing method is not particularly limited. For example, use may be made of a melt blending method using mixing tools such as ribbon blenders, tumble mixers, Henschel mixers, or blending tools such as open rollers, kneaders, Banbury mixers, and extruders. These mixing methods can be used alone or in combination.

For the resin component (ii), the xanthene derivative compound may be used in an amount of, for instance, 1-80 parts by weight, preferably 5-60 parts by weight, and more preferably 20-60 parts by weight per 100 parts by weight of the resin.

The resin component may include an additive. Because the resin component contains a xanthene frame work derived from the xanthene derivative compound, it can improve the dispersibility of the additives.

The additive may be in a liquid phase at room temperature (for example, at temperatures around 15-25° C.) or in a solid form (e.g., granular solids). Additives can include fillers or reinforcements, colorants (dyes), conductive agents, flame retardants, plasticizers, lubricants, stabilizers (antioxidants, ultraviolet absorbers, heat stabilizers, etc.), releasing agents (natural waxes, synthetic waxes, straight-chain fatty acids or their metal salts, acid amides, esters, paraffins, etc.), antistatic agents, dispersants, flow control agents, leveling agents, antifoaming agents, surface modifiers (silane coupling agents, titanium coupling agents, etc.), stress reducers (silicone oil, silicone rubber, various plastic powders, various high-performance plastic powders, etc.), heat resistance improvers (sulfur compounds or polysilanes), carbon materials, and so on. These additives can be used individually or in combination.

Among these additives, fillers, coloring agents (for example, black pigments, red pigments, green pigments, blue pigments, etc.), flame retardants, and carbon materials are preferable. Furthermore, carbon materials that function as fillers or reinforcements, coloring agents, and conductive agents are also desirable.

The resin components can be prepared into molded articles by common molding methods, such as injection molding, injection compression molding, extrusion molding, transfer molding, blow molding, compression molding, and coating methods (spin coating, roll coating, curtain coating, dip coating, casting, etc.), depending on their form (resin pellets, coating compositions, etc.). Furthermore, the shape of the molded article may be two-dimensional structures (films, sheets, coatings (or thin films), plates, etc.) or three-dimensional structures (for example, pipes, rods, tubes, ladders, hollow products, etc.).

Another aspect of the present disclosure provides a polyurethane (co)polymer manufactured from the xanthene derivative compound according to an aspect of the present disclosure; and a polymer component containing a diisocyanate compound.

As used herein, the term “(co)polymer” is intended to encompass both homopolymers and copolymers, and the polymer means a homopolymer consisting of a single repeating unit, and the copolymer means a complex polymer containing two or more repeating units.

Herein, the term “(co)polymer” includes random (co)polymers, block (co)polymers, graft (co)polymers, and the like.

In one embodiment of the present disclosure, the diisocyanate compound contains an isocyanate group and reacts with the hydroxyl group of the xanthene derivative compound or an additional diol compound to form a urethane bond.

No particular limitations are imparted to the diisocyanate compound that is used for the production of polyurethane.

In one embodiment of the present disclosure, the diisocyanate compound is selected from a group consisting of methylene diphenyl diisocyanate (MDI), p-phenylene diisocyanate (PPDI), tolylene-2,4-diisocyanate (2,4-TDI), tolylene-2,6-diisocyanate (2,6-TDI), xylylene diisocyanate (XDI), 1,5-naphthalene diisocyanate (NDI), hexamethylene diisocyanate (HDI), 4,4′-methylene dicyclohexyl diisocyanate (H12MDI), 1,4-cyclohexane diisocyanate (CHDI), isophorone diisocyanate (IPDI), and 1,3-bis(isocyanatomethyl)cyclohexane (H6XDI).

A further aspect of the present disclosure provides a polycarbonate (co)polymer manufactured from the xanthene derivative compound according to an aspect of the present disclosure; and a polymerizable component containing a polycarbonate precursor.

In one embodiment of the present disclosure, the aforementioned polycarbonate precursor is represented by the following Chemical Formula:

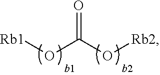

Wherein, Rb1 and Rb2 are same or different and are each independently a halogen; a substituted or unsubstituted alkyl; or a substituted or unsubstituted aryl, and
b1 and b2 are each independently 0 or 1.

The polycarbonate precursor can act to link additional comonomers as needed. Concrete examples include phosgene, triphosgene, diphosgene, bromophosgene, dimethyl carbonate, diethyl carbonate, dibutyl carbonate, dicyclohexyl carbonate, ditolyl carbonate, bis(chlorophenyl) carbonate, m-cresyl carbonate, dinaphthyl carbonate, bis(diphenyl) carbonate, and bishalophormate. These precursors may be used alone or in mixture.

The polymerization can be carried out by either interface polymerization or melt polymerization methods. So long as it is commonly used in the polymerization of polycarbonates in the industry, any solvent may be employed herein, without particular limitations thereto. For instance, halogenated hydrocarbons such as methylene chloride or chlorobenzene may be used.

Furthermore, it is preferable to carry out the polymerization in the presence of an acid-binding agent, and alkali metal hydroxides such as sodium hydroxide or potassium hydroxide, or amine compounds such as pyridine can be used as the acid-binding agent.

Also, for the purpose of controlling the molecular weight of the polycarbonate during polymerization, it is desirable to polymerize in the presence of a molecular weight control agent. C1-20 alkylphenols can be used as the molecular weight control agent, and concrete examples thereof include p-tert-butylphenol, p-cumylphenol, decylphenol, dodecylphenol, tetradecylphenol, hexadecylphenol, octadecylphenol, eicosylphenol, docosylphenol, and triacontylphenol. The molecular weight control agent may be added before, during, or after the initiation of polymerization.

Additionally, a tertiary amine compound such as triethylamine, tetrabutylammonium bromide, or tetrabutylphosphonium bromide, a quaternary ammonium compound, or a quaternary phosphonium compound may be further employed as a reaction catalyst to promote the polymerization reaction.

Another aspect of the present disclosure provides an optical lens comprising the polymer or copolymer according to an aspect of the present disclosure.

The optical lens can be manufactured in a desired shape by injecting the polymer or copolymer. In addition to injection, other processing methods can also be applied.

In an embodiment of the present disclosure, the polymer or copolymer that can be used to produce the optical lens has a high transmittance and heat resistance, which offers better processability compared to conventional lens materials, enabling mass production of plastic lenses through injection.

### Advantageous Effects of Invention

Provided herein is a compound that has a xanthene-based complex cardo structure and a high refractive index and can be used as a monomer in optical resins requiring a refractive index of 1.7 or higher, and a manufacturing method therefor. Given, the xanthene-based complex cardo structure of the compound suppresses the fluidity of the molecular chains and can find applications in the production of resins with high glass transition temperature and excellent thermal stability.

### BRIEF DESCRIPTION OF DRAWINGS

FIG. 1 is a flow chart illustrating a synthesis method for a xanthene derivative compound according to a first embodiment of the present disclosure.

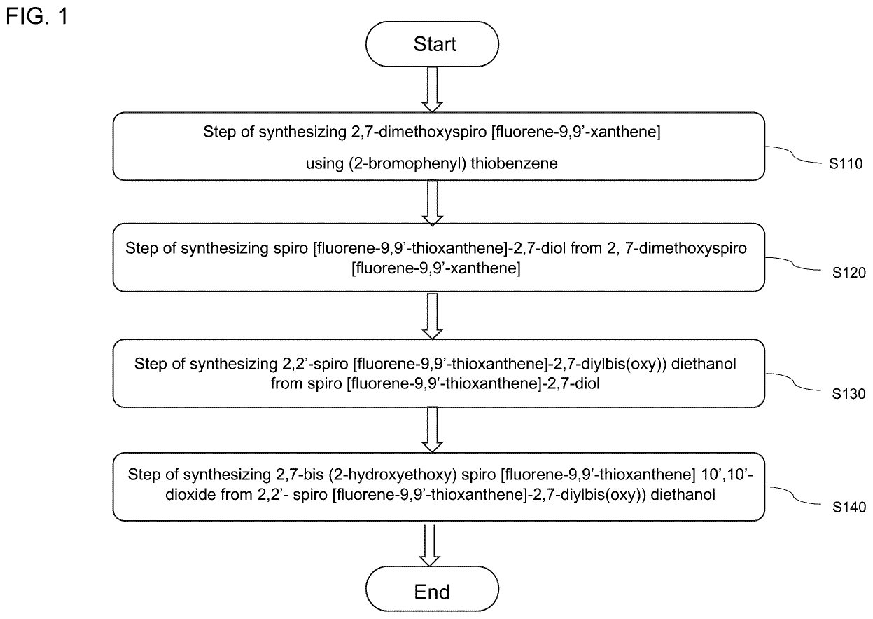

FIG. 2 is a flow chart illustrating a synthesis method for a xanthene derivative compound according to a second embodiment of the present disclosure.

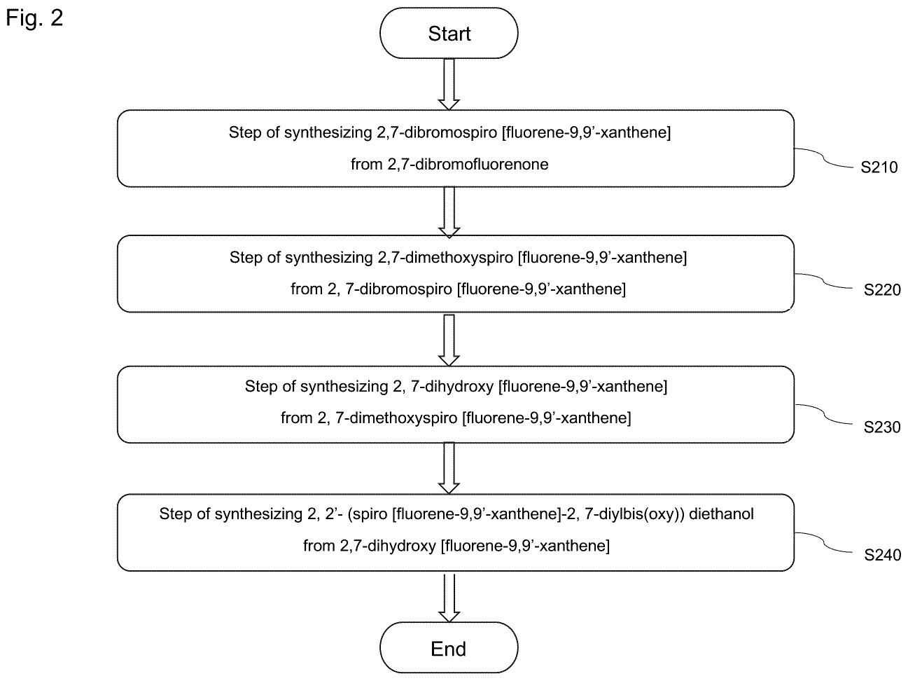

FIG. 3 is a view showing optical properties of the polyurethane materials PU-1 to PU-4 synthesized in Example 4 in terms of refractive index.

FIG. 4 is a view showing optical properties of the polyurethane materials PU-1 to PU-4 synthesized in Example 4 in terms of transmittance.

FIG. 5 is a view showing thermal properties of the polyurethane materials PU-1 to PU-4 synthesized in Example 4, as analyzed by differential scanning colorimetry (DSC) and thermogravimetric analysis (TGA).

FIG. 6 is a view showing optical properties of the polycarbonate materials PC-1 to PC-4 synthesized in Example 5 in terms of refractive index.

FIG. 7 is a view showing optical properties of the polycarbonate materials PC-1 to PC-4 synthesized in Example 5 in terms of transmittance.

FIG. 8 is a view showing thermal properties of the polycarbonate materials PC-1 to PC-4 synthesized in Example 5, as analyzed by differential scanning colorimetry (DSC) and thermogravimetric analysis (TGA).

### BEST MODE FOR CARRYING OUT THE INVENTION

Hereinafter, the present disclosure will be described in more detail through examples. These examples are only for illustrating the present disclosure in more detail, and it will be apparent to those skilled in the art that the scope of the present disclosure is not limited by these examples according to the gist of the present disclosure.

### EXAMPLES

Unless otherwise stated, “%” used to indicate the concentration of a specific substance is (weight/weight) % for solid/solid, (weight/volume) % for solid/liquid, and (volume/volume) olo for liquid/liquid throughout the specification.

### [Example 1] Synthesis of Novel Xanthene Derivative Compound 1

(1) Synthesis of 2,7-dimethoxyspiro[fluorene-9,9′-thioxanthene]

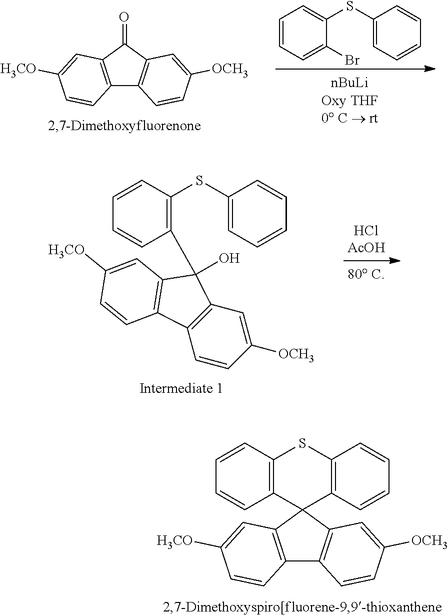 [SMILES: COc1ccc2c(c1)C(=O)c1cc(OC)ccc1-2 ; Brc1ccccc1Sc1ccccc1 ; COc1ccc2c(c1)C(O)(c1ccccc1Sc1ccccc1)c1cc(OC)ccc1-2 ; COc1ccc2c(c1)C1(c3ccccc3Sc3ccccc31)c1cc(OC)ccc1-2]

To a two-neck flask equipped with a thermometer and a stirrer was introduced (2-bromophenyl) thiobenzene (7.857 g, 29.6 mmol). Dry THF (15 ml) was added, followed by stirring. While the temperature was maintained at −78° C., 1.5M nBuLi (18 ml) was dropwise added. At the same temperature, stirring was conducted for one hour. To a different two-neck flask equipped with a stirrer were fed 2,7-dimethoxyfluorenone (5.2 g, 21.8 mmol) and dry THF (70 ml) which were then stirred together. This mixture was introduced into the flask maintained at −78° C. with the aid of a syringe. At the same temperature, stirring was conducted for one hour. Thereafter, the temperature was slowly increased to room temperature while stirring for 3 hours. The reaction mixture was subjected at a reduced pressure to extraction with chloroform and NaCl. The organic layer thus formed was isolated and left in a vacuum to obtain intermediate 1 (7.69 g, yield 83%).

In a two-neck equipped with a thermometer, a stirrer, and a condenser, intermediate 1 (7.69 g, 18 mmol) was stirred, together with acetic acid (70 ml) and 36% HCl (10 ml), at 80° C. for 10 hours. Neutralization of remaining HCl with NaHCO₃ and water was followed by filtration. The solid thus filtered was extracted with chloroform and NaHCO₃. The organic layer was dried in a vacuum and purified to afford compound 1 as a solid (7.15 g, yield 80%).

(2) Synthesis of Spiro[fluorene-9,9′-thioxanthene]-2,7-diol

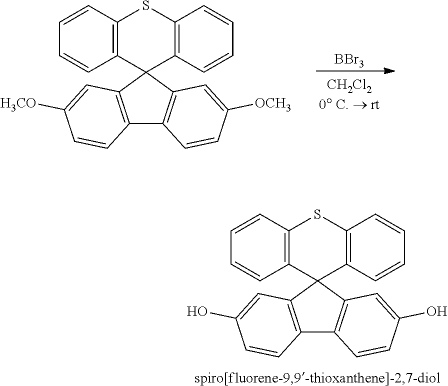 [SMILES: COc1ccc2c(c1)C1(c3ccccc3Sc3ccccc31)c1cc(OC)ccc1-2 ; "BBr3" ; Oc1ccc2c(c1)C1(c3ccccc3Sc3ccccc31)c1cc(O)ccc1-2]

To a two-neck flask equipped with a thermometer and a stirrer were introduced 2,7-dimethoxyspiro [fluorene-9,9′-thioxanthene] (3 g, 7.34 mmol) and dichloromethane (60 ml, 0.12 M) which were then stirred. After the temperature was lowered to 0° C., 1.0 M in BBr₃ dichloromethane (22 ml, 22.02 mmol) was dropwise added slowly. The mixture was stirred for 3 hours while the temperature was slowly increased to room temperature. Subsequently, the temperature was reduced to 0° C. and ice was introduced to quench BBr₃, followed by extraction with dichloromethane and NaHCO₃. The organic layer thus formed was isolated, dried in a vacuum, and purified to afford spiro[fluorene-9,9′-thioxanthene]-2,7-diol as a solid (2.7 g, yield 97%, purity 99.8%).

(3) Synthesis of 2,2′-(spiro[fluorene-9,9′-thioxanthene]-2,7-diylbis(oxy))diethanol (FTX Synthesis)

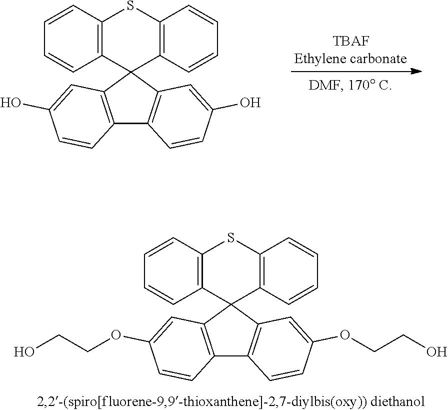 [SMILES: Oc1ccc2c(c1)C1(c3ccccc3Sc3ccccc31)c1cc(O)ccc1-2 ; OCCOc1ccc2c(c1)C1(c3ccccc3Sc3ccccc31)c1cc(OCCO)ccc1-2]

In a two-neck flask equipped with a thermometer and a stirrer were introduced spiro[fluorene-9,9′-thioxanthene]-2,7-diol (2.5 g, 6.57 mmol) and DMF (13 ml), followed by stirring. Ethylene carbonate (1 ml, 15.77 mmol) and TBAF (0.2 ml, 0.2 mmol) were added and stirring was conducted at 150° C. for 3 hours. After completion of the reaction, the temperature was decreased to room temperature and extraction was conducted with chloroform and water. The organic layer thus formed was isolated, dried in a vacuum, and purified to afford 2,2′-(spiro[fluorene-9,9′-thioxanthene]-2,7-diylbis(oxy))diethanol as a solid (2.83 g, yield 92%, purity 99.6%).

(4) Synthesis of 2,7-bis(2-hydroxyethoxy)spiro[fluorene-9,9′-thioxanthene]10′,10′-dioxide (FTXDO Synthesis)

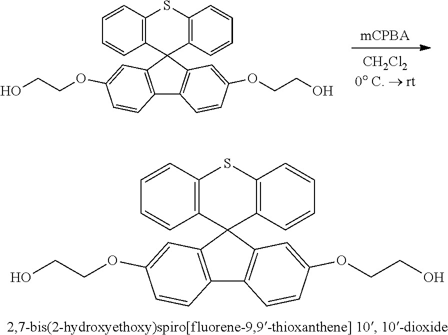 [SMILES: OCCOc1ccc2c(c1)C1(c3ccccc3Sc3ccccc31)c1cc(OCCO)ccc1-2 ; OCCOc1ccc2c(c1)C1(c3ccccc3Sc3ccccc31)c1cc(OCCO)ccc1-2]

To a two-neck flask equipped with a thermometer and a stirrer were introduced 2,2′-(spiro[fluorene-9,9′-thioxanthene]-2,7-diylbis(oxy))diethanol (105 mg, 0.22 mmol) and dichloromethane (7 ml, 0.03 M). The temperature was reduced to 0° C. and mCPBA (101 mg, 0.44 mmol) was added. While the temperature was slowly increased to room temperature, stirring was conducted for 5 hours.

After completion of the reaction, extraction was conducted with dichloromethane and NaHCO₃. The organic layer thus formed was isolated and concentrated. The concentrate was purified by column chromatography (eluents: ethyl acetate and hexane) to afford 2,7-bis(2-hydroxyethoxy)spiro[fluorene-9,9′-thioxanthene] 10′,10′-dioxide as a solid (83 mg, yield 75%, purity 99.2%).

### [Example 2] Synthesis of Novel Xanthene Derivative Compound 2

(1) Synthesis of 2,7-dibromospiro[fluorene-9,9′-xanthene]

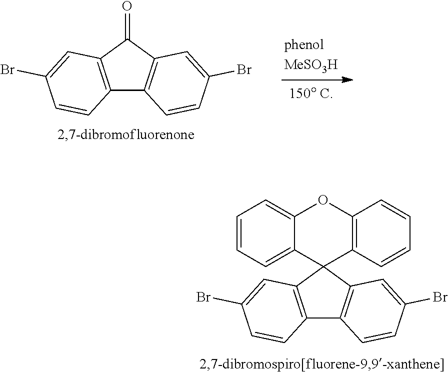

In a two-neck flask equipped with a thermometer and a stirrer, 2,7-dibromofluorenone (30 g, 88.76 mmol) and phenol (83 g, 887.6 mmol) were stirred together. Then, methanesulfonic acid (24 ml, 355.04 mmol) was added and stirring was conducted at 150° C. for 12 hours. After completion of the reaction, the reaction mixture was cooled to room temperature and subjected to extraction with chloroform, NaHCO₃, and NaCl. The organic layer thus formed was isolated, concentrated in a vacuum, and purified to afford compound 1 as a solid (33.28 g, yield 76.5%, purity 99.8%).

(2) Synthesis of 2,7-dimethoxyspiro[fluorene-9,9′-xanthene]

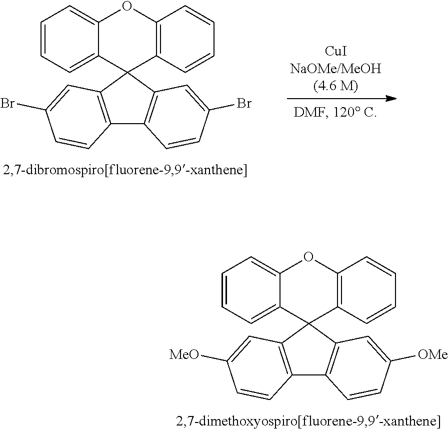 [SMILES: Brc1ccc2c(c1)C1(c3ccccc3Oc3ccccc31)c1cc(Br)ccc1-2 ; COc1ccc2c(c1)C1(c3ccccc3Oc3ccccc31)c1cc(OC)ccc1-2]

In a two-neck flask equipped with a thermometer, a stirrer, and a condenser, compound 1 (1 g, 2.039 mmol), CuI (1.55 g, 8.159 mmol), and dry DMF (3.3 ml) were stirred under a nitrogen atmosphere. NaOMe (14.7 ml, 4.6 M) was added before stirring at 120ºC for 24 hours under reflux. After completion of the reaction, the reaction mixture was diluted with chloroform and subjected to extraction with NH₄Cl and water. The organic layer was separated, concentrated in a vacuum, and purified to afford compound 2 as a solid (0.7 g, yield 87%, purity 99.7%).

(3) Synthesis of 2,7-dihydroxyspiro[fluorene-9,9′-xanthene]

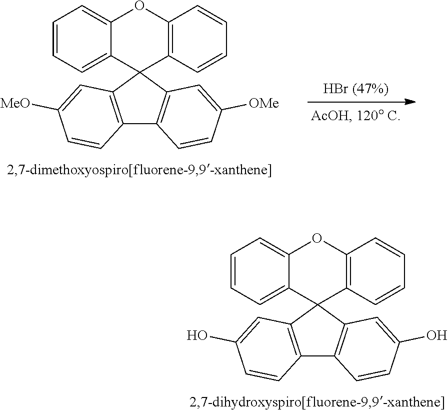 [SMILES: COc1ccc2c(c1)C1(c3ccccc3Oc3ccccc31)c1cc(OC)ccc1-2 ; Oc1ccc2c(c1)C1(c3ccccc3Oc3ccccc31)c1cc(O)ccc1-2]

In a two-neck flask equipped with a thermometer, a stirrer, and a condenser, compound (2 g, 5.096 mmol) was left under a nitrogen atmosphere. Glacial acetic acid (13 ml, 0.4 M) and 47% HBr (2.49 ml, 45.86 mmol) were added before stirring at 120° C. for 48 hours under reflux. After completion of the reaction, the reaction mixture was cooled to room temperature and subjected to extraction with chloroform and NaHCO₃. The organic layer thus formed was separated, concentrated in a vacuum, and purified to afford compound 3 as a solid (1.8 g, yield 97%, purity 99.5%).

(4) Synthesis of 2,2′-(spiro[fluorene-9,9′-xanthene]-2,7-diylbis(oxy))diethanol (FX Synthesis)

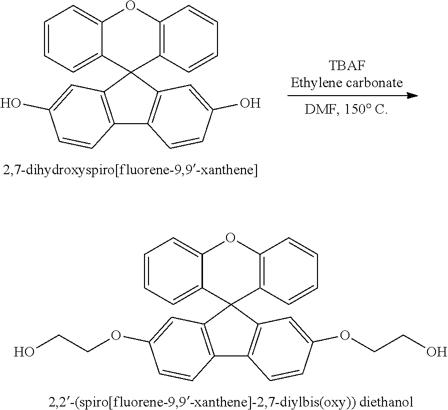 [SMILES: Oc1ccc2c(c1)C1(c3ccccc3Oc3ccccc31)c1cc(O)ccc1-2 ; OCCOc1ccc2c(c1)C1(c3ccccc3Oc3ccccc31)c1cc(OCCO)ccc1-2]

In a two-neck flask equipped with a thermometer, a stirrer, and a condenser, compound 3 (2 g, 5.488 mmol), dry DMF (13 ml), ethylene carbonate (0.887 ml, 12.07 mmol), and TBAF (0.1 ml, 0.1 mmol) were stirred together 150° C. for 3 hours under reflux. After completion of the reaction, the reaction mixture was cooled to room temperature and subjected to extraction with ethyl acetate and water. The organic layer thus formed was separated, concentrated in a vacuum, and purified to afford compound 4 as a solid (2.0 g, yield 83.3%, purity 99.3%).

### [Experimental Example 1] ¹H NMR (in CDCl₃) and HPLC Analysis of Compounds Synthesized in Example 1

¹H NMR (in CDCl₃) spectrum of the 2,7-dimethoxyspiro[fluorene-9,9′-thioxanthene] synthesized in Example 1-(1) is as follows:

### 2,7-Dimethoxyspiro[fluorene-9,9′-thioxanthene]

¹H-NMR (500 MHz, CDCl₃) : δ=7.60 (d, J=8.4 Hz, 2H), 7.42 (dd, J₁=7.8 Hz, J₂=1.2 Hz, 2H), 7.17 (td, J₁=7.5 Hz, J₂=1.4 Hz, 2H), 7.12 (d, J=2.4 Hz, 2H), 6.90-6.94 (m, 2H), 6.60 (dd, J₁=8.0 Hz, J₂=1.2 Hz, 2H), 3.74 (s, 6H.

¹H NMR (in CDCl₃) spectrum of the spiro[fluorene-9,9′-thioxanthene]-2,7-diol synthesized in Example 1-(2) is as follows:

### Spiro[fluorene-9,9′-thioxanthene ]-2,7-diol

¹H-NMR (500 MHz, CDCl₃) : δ=7.56 (d, J=8.2 Hz, 2H), 7.41 (dd, J₁=7.8 Hz, J₂=1.0 Hz, 2H), 7.17 (td, J₁=7.5 Hz, J₂=1.3 Hz, 2H), 7.01 (d, J=2.3 Hz, 2H), 6.92 (td, J₁=7.6 Hz, J₂=1.3 Hz, 2H), 6.86 (dd, J₁=8.2 Hz, J₂=2.4 Hz, 2H), 6.60 (dd, J₁=8.0 Hz, J₂=1.0 Hz, 2H), 4.68 (s, 2H).

¹H NMR spectrum (in CDCl₃) of the 2,2′-(spiro[fluorene-9,9′-thioxanthene]-2,7-diylbis(oxy))diethanol (FTX) synthesized in Example 1-(3) is as follows:

### 2,2′-(Spiro[fluorene-9,9′-thioxanthene]-2,7-diylbis(oxy))bis(ethan-1-ol) [FTX]

¹H-NMR (500 MHz, CDCl₃) : δ=7.57 (d, J=8.4 Hz, 2H), 7.39 (dd, J₁=7.8 Hz, J₂=1.2 Hz, 2H), 7.15 (td, J₁=7.5 Hz, J₂=1.4 Hz, 2H), 7.10 (d, J=2.4 Hz, 2H), 6.87-6.91 (m, 2H), 6.57 (dd, J₁=8.0 Hz, J₂=1.2 Hz, 2H), 3.98-4.00 (m, 4H), 3.87-3.90 (m, 4H), 1.93 (t, J=6.2 Hz, 2H).

¹H NMR (in CDCl₃) spectrum of the 2,7-bis(2-hydroxyethoxy)spiro[fluorene-9,9′-thioxanthene] 10′,10′-dioxide (FTXDO) synthesized in Example 1-(4) is as follows:

### 2,7-bis(2-hydroxyethoxy)spiro[fluorene-9,9′-thioxanthene] 10′,10′-dioxide [FTXDO]

1H-NMR (500 MHz, CDCl₃) : δ=8.24-8.23 (d, J=7.9 Hz, 2H), 7.65-7.64 (m, 2H), 7.50-7.47 (t, J=7.6 Hz, 2H), 7.31-7.30 (t, J=7.7 Hz, 2H), 6.97-6.96 (m, 2H), 6.90 (s, 2H), 6.64-6.63 (d, J=8.1 Hz, 2H), 3.95-3.94 (m, 4H), 3.86-3.85 (m, 4H).

The synthesis method according to Example 1 was observed to guarantee high purity for the final product 2,7-bis(2-hydroxyethoxy)spiro[fluorene-9,9′-thioxanthene] 10′,10′-dioxide and its intermediates

### [Experimental Example 2] ¹H NMR (in CDCl₃) and HPLC Analysis of Compounds Synthesized in Example 2

¹H NMR (in CDCl₃) spectrum of the 2,7-dibromospiro[fluorene-9,9′-xanthene] synthesized in Example 2-(1) is as follows:

### 2,7-dibromospiro[fluorene-9,9′-xanthene]

1H-NMR (500 MHz, CDCl₃): δ=7.64-7.62 (m, 2H), 7.51-7.49 (dd, J=8.1 Hz, 1.5 Hz, 2H), 7.24-7.23 (d, J=3.8 Hz, 6H), 6.84-6.81 (dt, J=8.1 Hz, 4.1 Hz, 2H), 6.39-6.37 (m, J=7.8 Hz, 2H).

¹H NMR (in CDCl₃) spectrum of the 2,7-dimethoxyspiro[fluorene-9,9′-xanthene] synthesized in Example 2-(2) is as follows:

### 2,7-dimethoxyspiro [fluorene-9,9′-xanthene]

1H-NMR (500 MHz, CDCl₃): δ=7.60-7.58 (d, J=8.4 Hz, 2H), 7.22-7.19 (m, 4H), 6.89-6.87 (dd, J=8.4 Hz, 2.4 Hz, 2H), 6.81-6.78 (ddd, J=8 Hz, 6.4 Hz, 2 Hz, 2H), 6.66 (d, J=2.4 Hz, 2H), 6.46-6.44 (m, 2H), 3.69 (s, 6H).

¹H NMR (in CDCl₃) spectrum of the 2,7-dihydroxyspiro[fluorene-9,9′-xanthene] synthesized in Example 2-(3) is as follows:

### 2,7-dihydroxyspiro[fluorene-9,9′-xanthene]

¹H-NMR (500 MHz, CDCl₃) : δ=7.57 (d, J=8.2 Hz, 2H), 7.22-7.23 (m, 4H), 6.82-6.86 (m, 4H), 6.61 (d, J=2.3 Hz, 2H), 6.49 (d, J=7.6 Hz, 2H), 4.57 (s, 2H).

¹H-NMR (in CDCl₃) spectrum and HPLC chromatogram of the 2,2′-(spiro[fluorene-9,9′-xanthene]-2,7-diylbis(oxy)) diethanol (FX) synthesized in Example 2-(4) is as follows :

### 2,2′-(Spiro[fluorene-9,9′-xanthene]-2,7-diylbis(oxy))bis(ethan-1-ol) [FX]

¹H-NMR (500 MHz, CDCl₃) : δ=7.60 (d, J=8.2 Hz, 2H), 7.17-7.22 (m, 4H), 6.90 (dd, J₁=8.3 Hz, J₂=2.4 Hz, 2H), 6.77-7.82 (m, 2H), 6.68 (d, J=2.3 Hz, 2H), 6.44 (dd, J₁=7.8 Hz, J₂=1.4 Hz, 2H), 3.95-3.97 (m, 4H), 3.84-3.88 (m, 4H), 1.90 (t, J=6.2 Hz, 2H).

The synthesis method according to Example 2 was observed to guarantee high purity for the final product 2,2′-(spiro[fluorene-9,9′-xanthene]-2,7-diylbis(oxy))diethanol and its intermediates.

### [Experimental Example 3] Synthesis of Novel Xanthene Derivative Compound 3

2,7-Bis(2-hydroxy)spiro[fluorene-9,9′-thioxanthene] 10′,10′-dioxide was synthesized as illustrated in the following Reaction Scheme 3.

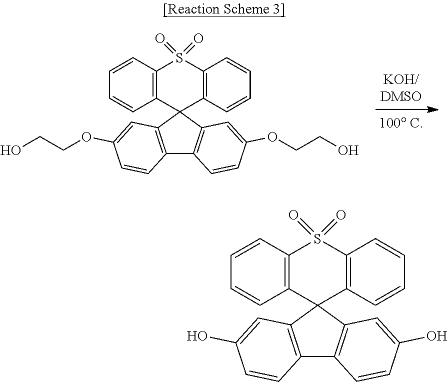 [SMILES: O=S1(=O)c2ccccc2C2(c3cc(OCCO)ccc3-c3ccc(OCCO)cc32)c2ccccc21 ; O=S1(=O)c2ccccc2C2(c3cc(O)ccc3-c3ccc(O)cc32)c2ccccc21]

In brief, 2,7-bis(2-hydroxyethoxy)spiro[fluorene-9,9′-thioxanthene] 10′,10′-dioxide (112 mg, 0.22 mmol) was introduced into a two-neck flask equipped with a thermometer and a stirrer. DMSO (1 ml) was added before stirring. Then, KOH (74 mg, 1.32 mmol) was added, followed by stirring at 100° C. for 6 hours. After completion of the reaction, the temperature was decreased to room temperature. The reaction mixture was adjusted to a pH of 3, using 1N HCl before extraction with ethyl acetate and water. The organic layer thus formed was separated, concentrated in a vacuum, and purified to afford 2,7-bis(2-hydroxy)spiro[fluorene-9,9′- thioxanthene] 10′,10′-dioxide (82 mg, yield 91.0%, purity 99.3%).

### [Experimental Example 3] ¹H NMR (in CDCl₃) and HPLC Analysis of Compounds Synthesized in Example 3

¹H NMR (in CDCl₃) spectrum of the 2,7-bis(2-hydroxy)spiro[fluorene-9,9′-thioxanthene] 10′,10′-dioxide synthesized in Example 3 is as follows:

### 2,7-bis(2-hydroxy)spiro[fluorene-9,9′-thioxanthene] 10′,10′-dioxide [FTXDO-OH]

¹H-NMR (500 MHz, CDCl₃): δ=5.02 (s, 2 H), 6.66 (d, J=8.24 Hz, 2 H), 6.75 (s, 2 H), 6.90 (d, J=2.44 Hz, 2 H), 7.32 (br. s., 1 H), 7.50 (s, 1 H), 7.60 (d, J=8.39 Hz, 2 H), 8.22 (d, J=8.09 Hz, 2 H).

The synthesis method according to Example 3 was observed to guarantee high purity for the final product 2,7-bis(2-hydroxy)spiro[fluorene-9,9′-thioxanthene] 10′,10′-dioxide.

### [Example 4] Synthesis of Polyurethane With Novel Xanthene-Based Monomer

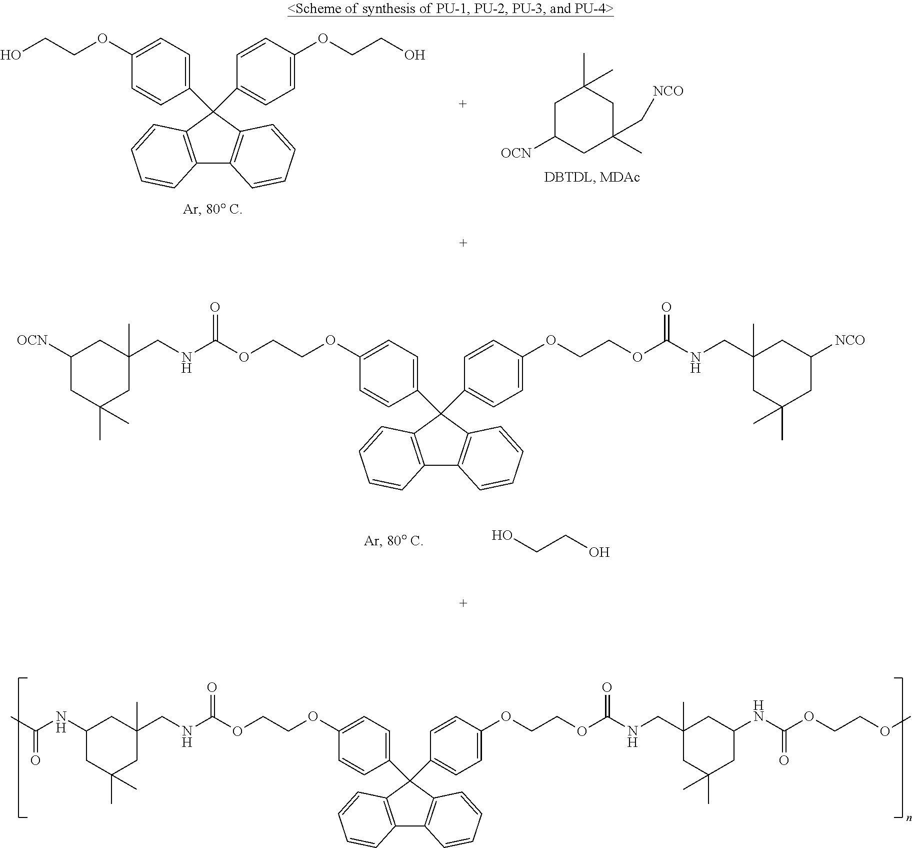

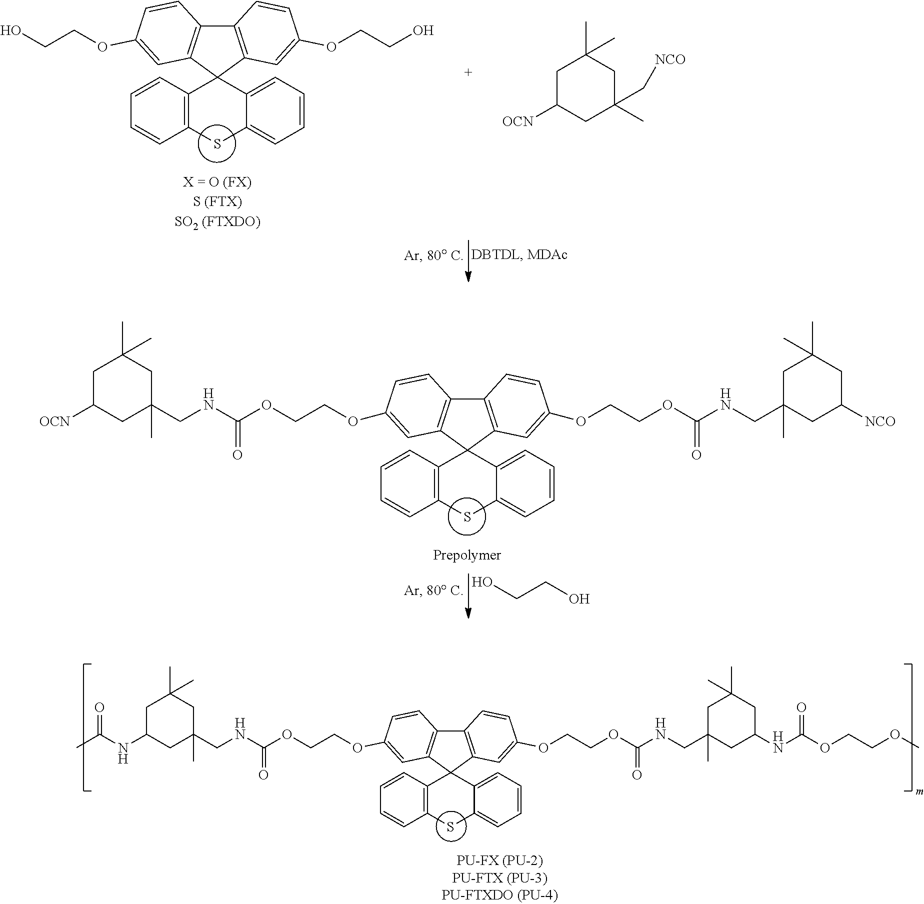

To a solution of 1.0 equiv. diol monomer compound in 0.46 M anhydrous DMAc was added 1.5 equiv. isophorone diisocyanate (IPDI), together with 4 mol % dibutyltin dilaurate (DBTDL) as a catalyst. The mixture was stirred at 80° C. under an argon atmosphere. After 3 hours, anhydrous ethylene glycol (1.5 equiv.) was added as a chain extender to the solution. The mixture was stirred at 80° C. for 3 hours under an argon atmosphere. The reaction mixture was cooled to room temperature and added with water to give a primary product as a precipitate. This product was dissolved in THE and precipitated again in water. The remaining solvent was removed by drying at room temperature in a vacuum to afford the final product.

### (1) Synthesis of PU-FBPE (PU-1)

PU-FBPE was synthesized from the conventional high-refractive index material 4,4′-(9-fluorenylidene)bis(2-phenoxyethanol) [FBPE] (4.00 g, 9.12 mmol) according to the general reaction scheme. The product was obtained as a white solid.

### (2) Synthesis of PU-FX (PU-2)

PU-FX was synthesized from 2,2′-(spiro[fluorene-9,9′-xanthene]-2,7-diylbis(oxy)) diethanol [FX] (2.06 g, 4.56 mmol) according to the general reaction scheme. The product was obtained as an off-white solid.

### (3) Synthesis of PU-FTX (PU-3)

PU-FTX was synthesized from 2,2′-(spiro[fluorene-9,9′-thioxanthene]-2,7-diylbis(oxy))diethanol [FTX] (2.14 g, 4.56 mmol) according to the general reaction scheme. The product was obtained as a white solid.

### (4) Synthesis of PU-FTXDO (PU-4)

PU-FTXDO was synthesized from 2,7-bis(2-hydroxyethoxy)spiro[fluorene-9,9′-thioxanthene] 10′,10′-dioxide [FTXDO] (2.28 g, 4.56 mmol) according to the general reaction scheme. The product was obtained as an off-white solid.

### [Experimental Example 4] Characterization of Polyurethane Synthesized in Example 4

### 4-1. Analysis for Refractive Index of Synthesized Polyurethane

The polyurethanes PU-1 to PU-4 synthesized in Example 4 were analyzed for refractive index.

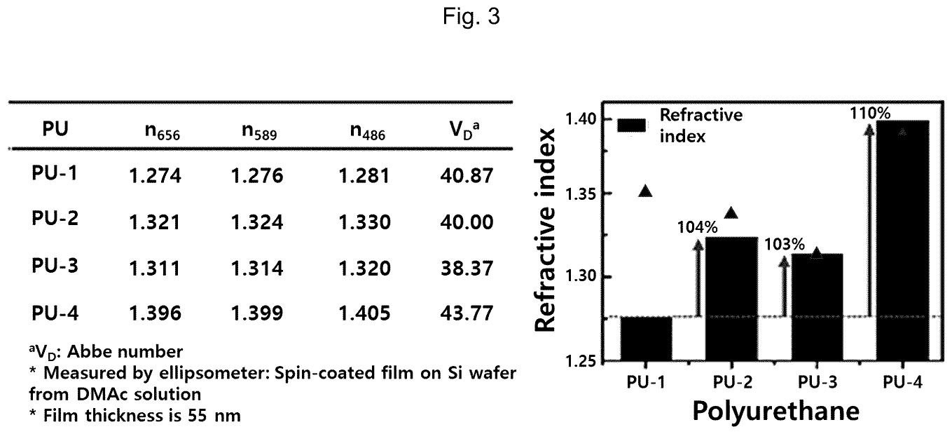

For use in measuring refractive indices, sample solutions in DMAc (dimethylacetamide) were prepared into films 55 μm thick on Si wafer by a spin coating method. Refractive indices of the films were measured using Spectroscopic Ellipsometer (Nano-View, SeMG-100). The measurements are depicted in FIG. 3.

As shown in FIG. 3, compared to PU-1 prepared using the conventional high-refractive index material 4,4′-(9-fluorenylidene)bis(2-phenoxyethanol) [FBPE] , the polyurethane materials PU-2 to PU-4 prepared from the high-refractive index monomers 2,2′-(spiro[fluorene-9,9′-xanthene]-2,7-diylbis(oxy)) diethanol [FX], 2,2′-(spiro[fluorene-9,9′-thioxanthene]-2,7-diylbis(oxy))diethanol [FTX], and 2,7-bis(2-hydroxyethoxy)spiro[fluorene-9,9′-thioxanthene] 10′,10′-dioxide [FTXDO], which were all newly synthesized in the present disclosure, were observed to have improved refractive indices.

### 4-2. Analysis for Transmittance of Synthesized Polyurethane

The polyurethanes PU-1 to PU-4 synthesized in Example 4 were analyzed for transmittance.

For use in measuring transmittance, sample solutions in DMAc (dimethylacetamide) were prepared into films 55 μm thick on slide glass by a spin coating method. Transmittance of the films was measured using UV-1800 spectrophotometer (Shimadzu).

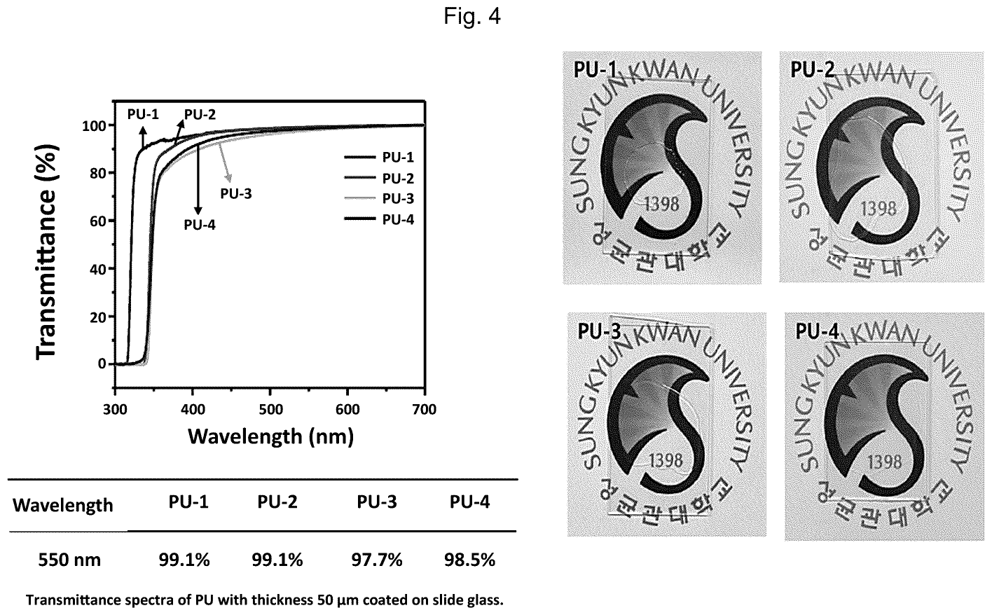

The measurements are depicted in FIG. 4.

As shown in FIG. 4, the novel polyurethane materials PU-2 to PU-4 synthesized in Example 4 of the present disclosure were all found to have excellent transmittance as in the polyurethane material PU-1 made using conventional monomers.

### 4-3. Analysis for Thermal Properties of Synthesized Polyurethane

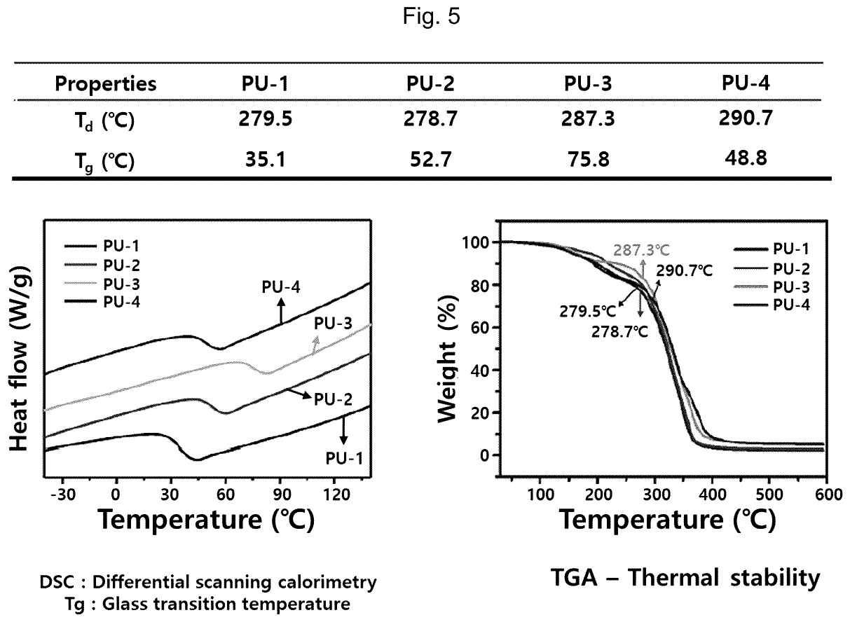

To analyze the thermal properties of the polyurethane materials PU-1 to PU-4 synthesized in Example 4, differential scanning calorimetry (DSC) and thermogravimetric analysis (TGA) were performed. The decomposition temperature (Td) and glass transition temperature (Tg) along with other measured analysis results are depicted in FIG. 5.

As shown in FIG. 5, the novel polyurethane materials PU-2 to PU-4 synthesized in Example 4 of the present disclosure all showed similar or even higher decomposition temperatures (Td) compared to the polyurethane material PU-1 made using conventional monomers, indicating superior thermal stability. Moreover, when compared to PU-1, there was a significant increase in the glass transition temperature, indicating excellent heat resistance.

### 4-4. Analysis for Molecular Weight of Synthesized Polyurethane

Furthermore, the polyurethane materials PU-1 to PU-4 synthesized in Example 4 were analyzed for molecular weight, and the results are summarized in Table 1.

TABLE 1

| Polyurethane | Mn | Mw | PDI |
|---|---|---|---|
| PU-1 | 16.3K | 32.5K | 1.99 |
| PU-2 | 18.3K | 36.9K | 2.01 |
| PU-3 | 52.2K | 96.3K | 1.92 |
| PU-4 | 33.0K | 66.8K | 2.01 |

Mn: number averaged molecular weight
Mw: weight averaged molecular weight
PDI: polydispersity index

### [Example 5] Synthesis of Polycarbonate with Novel Xanthene-Based Monomer

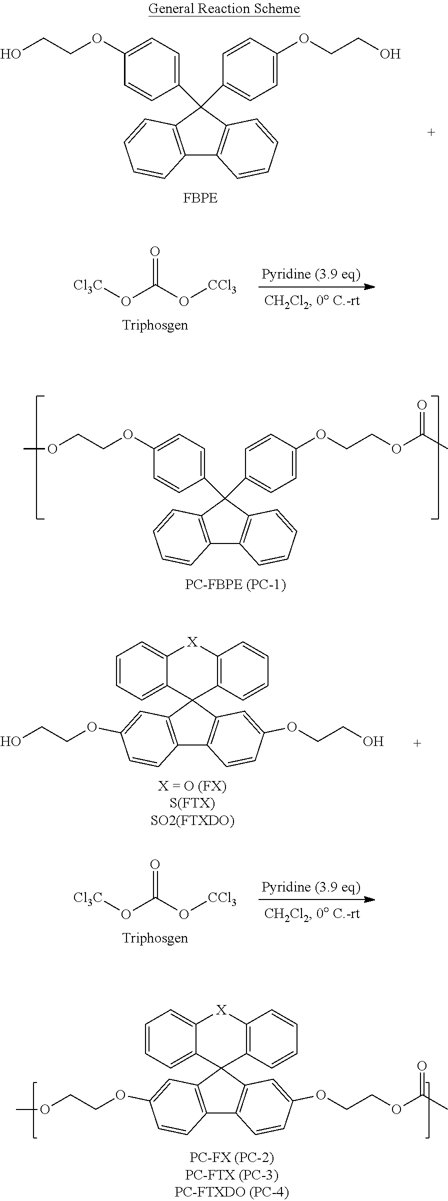

A solution of diol monomer compound (1.0 equiv.) and pyridine (3.9 equiv.) in DCM was cooled to 0° C. and slowly added with triphosgene (0.4 equiv.) over 2 hours while stirring. The mixture was stirred at 0° C. for 30 minutes and at room temperature for 5 hours. The reaction mixture was washed with water and the organic layer was concentrated to give a primary solid product. This solid product was precipitated in a mixture of isopropanol and water (9:1). Drying at room temperature in a vacuum removed the remaining solvent to afford the final products PC-1 to PC-4.

### (1) Synthesis of PC-FBPE (PC-1)

PC-FBPE was synthesized from the conventional high-refractive index monomer 4,4′-(9-fluorenylidene)bis(2-phenoxyethanol) [FBPE] (1.00 g, 2.28 mmol) and DCM (0.2 M) according to the general reaction scheme. The product was obtained as a white solid.

### (2) Synthesis of PC-FX (PC-2)

PC-FX was synthesized from 2,2′-(spiro[fluorene-9,9′-xanthene]-2,7-diylbis(oxy)) diethanol [FX] (1.00 g, 2.20 mmol) and DCM (0.05M) according to the general reaction scheme. The product was obtained as a white solid.

### (3) Synthesis of PC-FTX (PC-3)

PC-FTX was synthesized from 2,2′-(spiro[fluorene-9,9′-thioxanthene]-2,7-diylbis(oxy))diethanol [FTX] (1.00 g, 2.12 mmol) and DCM (0.05M) according to the general reaction scheme. The product was obtained as a white solid.

### (4) Synthesis of PC-FTXDO (PC-4)

PC-FTXDO was synthesized from FTXDO (1.00 g, 1.99 mmol) and DCM (0.17 M) according to the general reaction scheme. The product was obtained as a white solid.

### [Experimental Example 5] Characterization of Polycarbonate Synthesized in Example 5

### 5-1. Analysis for Refractive Index of Synthesized Polycarbonate

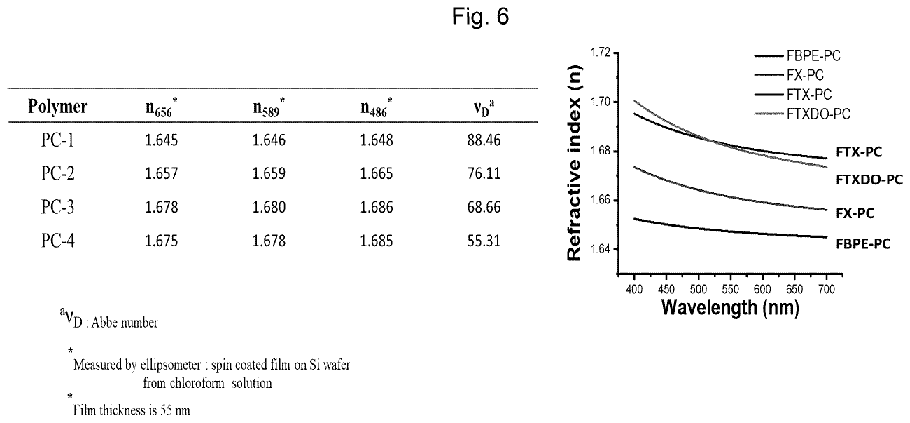

The polycarbonate PC-1 to PC-4 synthesized in Example 5 were analyzed for refractive index. The results are depicted in FIG. 6.

As shown in FIG. 6, compared to the polycarbonate PC-1 prepared using the conventional high-refractive index 4,4′-(9-fluorenylidene)bis(2-phenoxyethanol) material [FBPE], the polycarbonates PC-2 to PU-4 prepared from the high-refractive index monomers 2,2′-(spiro[fluorene-9,9′-xanthene]-2,7-diylbis(oxy)) diethanol [FX], 2,2′-(spiro[fluorene-9,9′-thioxanthene]-2,7-diylbis(oxy))diethanol [FTX], and 2,7-bis(2-hydroxyethoxy)spiro[fluorene-9,9′-thioxanthene] 10′,10′-dioxide [FTXDO], which were all newly synthesized in the present disclosure, were observed to have improved refractive indices.

### 5-2. Analysis for Transmittance of Synthesized Polycarbonate

The polycarbonates PC-1 to PC-4 synthesized in Example 4 were analyzed for transmittance in the same manner as in Experimental Example 4-2.

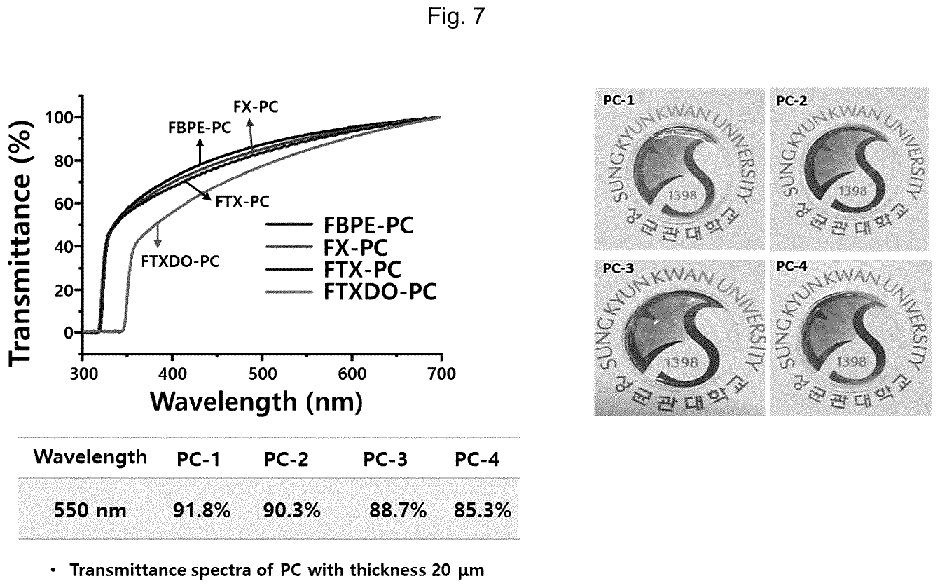

The measurements are depicted in FIG. 7.

As shown in FIG. 7, the novel polycarbonate materials PC-2 to PC-4 synthesized in Example 5 of the present disclosure were all found to have excellent transmittance as in the polycarbonate material PC-1 made using conventional monomers.

### 5-3. Analysis for Thermal Properties of Synthesized Polycarbonate

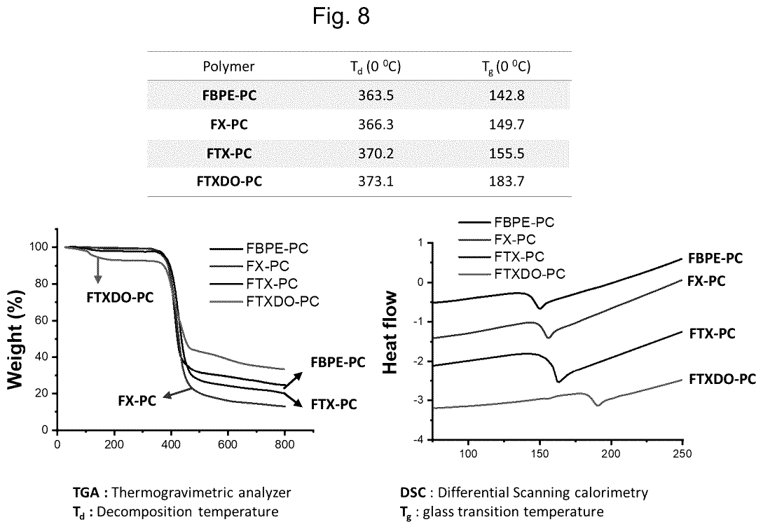

To analyze the thermal properties of the polycarbonate materials PC-1 to PC-4 synthesized in Example 5, differential scanning calorimetry (DSC) and thermogravimetric analysis (TGA) were performed. The decomposition temperature (Td) and glass transition temperature (Tg) along with other measured analysis results are depicted in FIG. 8.

As shown in FIG. 8, the novel polycarbonate materials PC-2 to PC-4 synthesized in Example 5 of the present disclosure all showed similar or even higher decomposition temperatures (Td) compared to the polycarbonate material PC-1 made using conventional monomers, indicating superior thermal stability. Moreover, when compared to PC-1, there was a significant increase in the glass transition temperature, indicating excellent heat resistance.

### 5-4. Analysis for Molecular Weight of Synthesized Polycarbonate

The results are summarized in Table 2.

TABLE 2

| Polycarbonate | Mn | Mw | PDI |
|---|---|---|---|
| PC-1 | 15.5K | 30.8K | 2.20 |
| PC-2 | 10.9K | 40.8K | 3.70 |
| PC-3 | 6.8K | 31.4K | 4.50 |
| PC-4 | 8.2K | 29.6K | 3.60 |

Mn: number averaged molecular weight
Mw: weight averaged molecular weight
PDI: polydispersity index

While the embodiments of the present disclosure and their advantages have been described in detail above, it should be understood that various changes, substitutions and alterations may be made herein without departing from the scope of the disclosure.

1. A compound having a chemical structure of the following Chemical Formula 1: 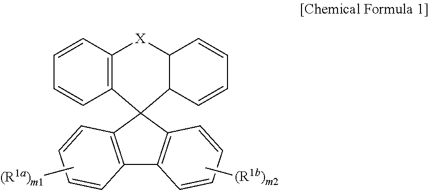
wherein X, R^{1a}, and R^{1b} represent substituents;
X is O, S, or SO₂;
m1 and m2 are each independently an integer of 0 to 4 (with a proviso that m1+m2 is an integer of 1 to 8),
R^{1a} and R^{1b} are each selected from the substituents having the chemical structures of Chemical Formulas 1-1 to 1-3, below: 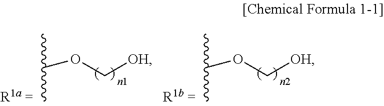
wherein, n1 and n2 are each independently an integer of 1 to 5, with a proviso that n1+n2 is 2 to 10; 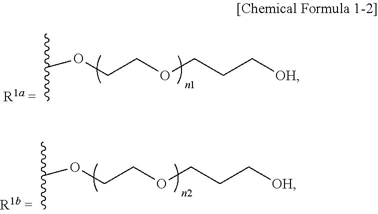
wherein, n1 and n2 are each independently an integer of 1 to 5, with a proviso that n1+n2 is 2 to 10; and

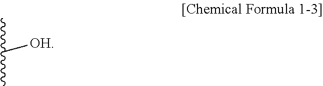 [SMILES: *O]

2. The compound of claim 1, wherein the compound has any one of the chemical structures of the following Chemical Formulas 2-1 to 2-3: 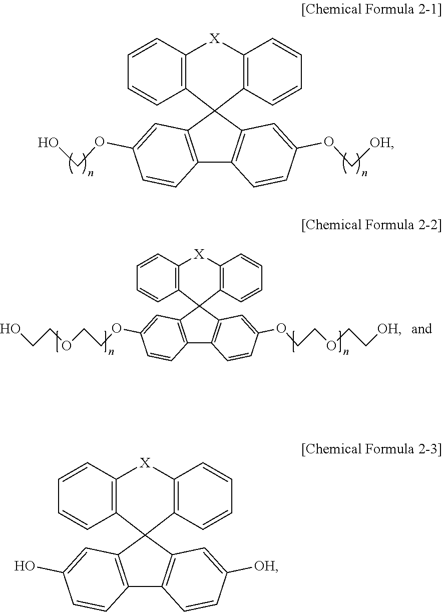
wherein,
X is O, S, or SO₂, and
n is an integer of 1 to 5.

3. The compound of claim 1, wherein X is SO₂.

4. The compound of claim 3, wherein the compound has the chemical structure of the following Chemical Formula 3-1 or 3-2:

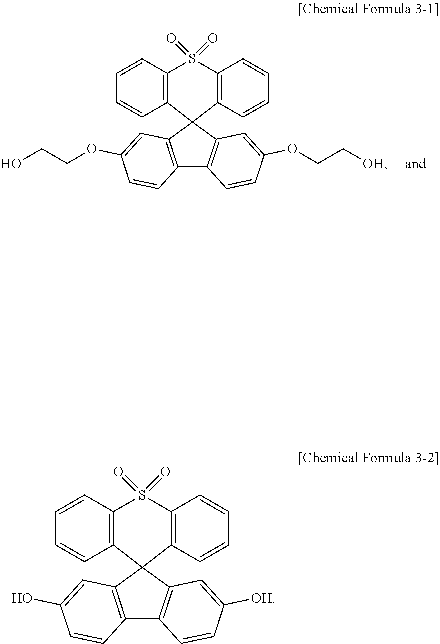 [SMILES: O=S1(=O)c2ccccc2C2(c3cc(OCCO)ccc3-c3ccc(OCCO)cc32)c2ccccc21 ; "and" ; O=S1(=O)c2ccccc2C2(c3cc(O)ccc3-c3ccc(O)cc32)c2ccccc21]

5. The compound of claim 1, wherein X is O.

6. The compound of claim 5, wherein the compound has the chemical structure of the following Chemical Formula 4-1 or 4-2

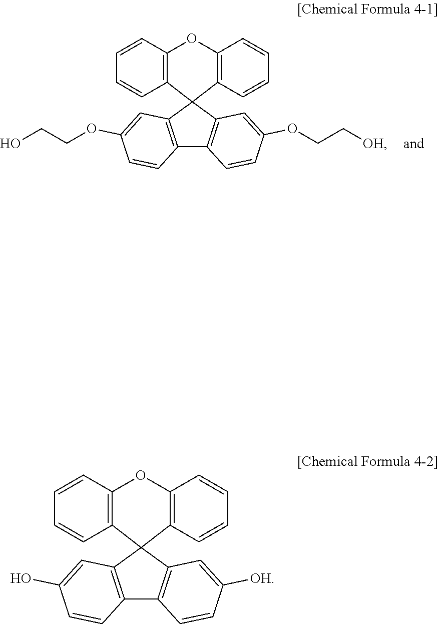 [SMILES: OCCOc1ccc2c(c1)C1(c3ccccc3Oc3ccccc31)c1cc(OCCO)ccc1-2 ; "and" ; Oc1ccc2c(c1)C1(c3ccccc3Oc3ccccc31)c1cc(O)ccc1-2]

7. The compound of claim 1, wherein X is S.

8. The compound of claim 7, wherein the compound has the chemical structure of the following Chemical Formula 5-1 or 5-2:

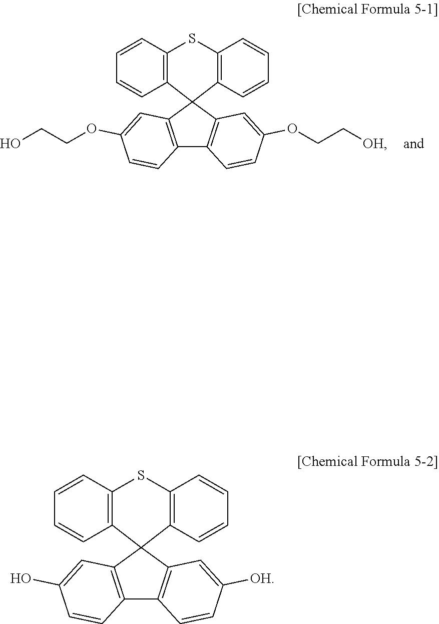 [SMILES: OCCOc1ccc2c(c1)C1(c3ccccc3Sc3ccccc31)c1cc(OCCO)ccc1-2 ; "and" ; Oc1ccc2c(c1)C1(c3ccccc3Sc3ccccc31)c1cc(O)ccc1-2]

9. A (co)polymer, prepared from the polymerization components comprising:
(a) the compound of claim 1; and
(b) a diisocyanate compound or a polycarbonate precursor.

10. The (co)polymer of claim 9, wherein the diisocyanate compound is at least one selected from the group consisting of methylene diphenyl diisocyanate (MDI), p-phenylene diisocyanate (PPDI), tolylene-2,4-diisocyanate (2,4-TDI), tolylene-2,6-diisocyanate (2,6-TDI), xylylene diisocyanate (XDI), 1,5-naphthalene diisocyanate (NDI), hexamethylene diisocyanate (HDI), 4,4′-methylene dicyclohexyldiisocyanate (H12MDI), 1,4-cyclohexane diisocyanate (CHDI), isophoroene diisocyanate (IPDI), and 1,3-bis(isocyanatomethyl) cyclohexane (H6XDI).

11. The (co)polymer, wherein the polycarbonate precursor is represented by the following Chemical Formula: 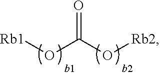
wherein, Rb1 and Rb2 are same or different and are each independently a halogen, a substituted or unsubstituted alkyl, or a substituted or unsubstituted aryl, and
b1 and b2 are each independently 0 or 1.

12. An optical lens, comprising the (co)polymer of claim 9.
